(19) United States
(12) Patent Application Publication
(10) Pub. No.: US 2004/0019046 A1
(43) Pub. Date: Jan. 29, 2004

(54) Phenylacetamido-pyrazole derivatives and their use as antitumor agents

(76) Inventors: Paolo Pevarello, Pavia (IT); Paolo Orsini, Gallarate-Varese (IT); Gabriella Traquandi, Milano (IT); Gabriella Brasca, Cusago (IT); Raffaella Amici, Piacenza (IT); Manuella Villa, Lurago (IT); Claudia Piutti, Cantone di Nerviano (IT); Mario Varasi, Milano (IT); Antonio Longo, Milano (IT)

Correspondence Address: Akin Gump Straus Hauer & Feld, 711 Louisiana, Suite 1900, Houston, TX, 77002, US

(21) Appl. No.: 10/432,119

(22) PCT Filed: Nov. 22, 2001
(86) PCT No.: PCT/EP01/13617

(51) Int. Cl. A61K 31/541; A61K 31/5377; A61K 31/496; A61K 31/454; A61K 31/427; A61K 31/422; A61K 31/416

(52) U.S. Cl. 514/227.8; 514/238.8; 514/254.05; 514/326; 514/406; 514/365; 514/374

(57) ABSTRACT

Phenylacetamido-pyrazoles and, more particularly, N-(5-cycloalkyl-1H-pyrazol-3-yl)phenylacetamido derivatives, optionally further substituted as reported in the description; or pharmaceutically acceptable salts thereof; are useful in the treatment of cell proliferative disorders, e.g. cancer, associated with an altered cell cycle dependent kinase activity.

### BACKGROUND OF THE INVENTION

1. Field of the Invention

The present invention relates to phenylacetamido-pyrazole derivatives, to a process for their preparation, to pharmaceutical compositions comprising them, and to their use as therapeutic agents, particularly in the treatment of cancer and cell proliferative disorders.

2. Discussion of the Background

Several cytotoxic drugs such as, e.g., fluorouracil (5-FU), doxorubicin and camptothecins, damage DNA or affect cellular metabolic pathways and thus cause, in many cases, an indirect block of the cell cycle. Therefore, by producing an irreversible damage to both normal and tumor cells, these agents result in a significant toxicity and side-effects.

In this respect, compounds capable of functioning as highly specific antitumor agents by selectively leading to tumor cell arrest and apoptosis, with comparable efficacy but reduced toxicity than the currently available drugs, are desirable.

It is well known that progression through the cell cycle is governed by a series of checkpoint controls, otherwise referred to as restriction points, which are regulated by a family of enzymes known as the cyclin-dependent kinases (cdk). In turn, the cdks themselves are regulated at many levels such as, for instance, binding to cyclins.

The coordinated activation and inactivation of different cyclin/cdk complexes is necessary for normal progression through the cell cycle. Both the critical G1-S and G2-M transitions are controlled by the activation of different cyclin/cdk activities. In G1, both cyclin D/cdk4 and cyclin E/cdk2 are thought to mediate the onset of S-phase. Progression through S-phase requires the activity of cyclin A/cdk2 whereas the activation of cyclin A/cdc2 (cdk1) and cyclin B/cdc2 are required for the onset of metaphases. For a general reference to cyclins and cyclin-dependent kinases see, for instance, Kevin R. Webster et al, in Exp. Opin. Invest. Drugs, 1998, Vol. 7(6), 865-887. Checkpoint controls are defective in tumor cells due, in part, to disregulation of cdk activity. For example, altered expression of cyclin E and cdks has been observed in tumor cells, and deletion of the cdk inhibitor p27 KIP gene in mice has been shown to result in a higher incidence of cancer.

Increasing evidence supports the idea that the cdks are rate-limiting enzymes in cell cycle progression and, as such, represent molecular targets for therapeutic intervention. In particular, the direct inhibition of cdk/cyclin kinase activity should be helpful in restricting the unregulated proliferation of a tumor cell.

### SUMMARY OF THE INVENTION

It is an object of the invention to provide compounds which are useful in treating cell proliferative disorders caused by and/or associated with an altered cell cycle dependent kinase activity. It is another object to provide compounds which have cdk/cyclin kinase inhibitory activity.

The present inventors have now discovered that certain phenylacetamido-pyrazoles are endowed with cdk/cyclin kinase inhibitory activity and are thus useful in therapy as antitumor agents and lack, in terms of both toxicity and side effects, the aforementioned drawbacks associated with currently available antitumor drugs.

More specifically, the phenylacetamido-pyrazoles of the invention are useful in the treatment of a variety of cancers including, but not limited to: carcinoma such as bladder, breast, colon, kidney, liver, lung, including small cell lung cancer, esophagus, gall-bladder, ovary, pancreas, stomach, cervix, thyroid, prostate, and skin, including squamous cell carcinoma; hematopoietic tumors of lymphoid lineage including leukemia, acute lymphocitic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias, myelodysplastic syndrome and promyelocytic 1 eukemia; tumors of mesenchymal origin, including fibrosarcoma and rhabdomyosarcoma; tumors of the central and peripheral nervous system, including astroc)toma neuroblastoma, glioma and schwannomas; other tumors, including melanoma, seminoma, teratocarcinoma, osteosarcoma, xeroderma pigmentosum, keratoxanthoma, thyroid follicular cancer and Kaposi's sarcoma.

Due to the key role of cdks in the regulation of cellular proliferation, the carbonylamino-pyrazole derivatives are also useful in the treatment of a variety of cell proliferative disorders such as, for example, benign prostate hyperplasia, familial adenomatosis polyposis, neurofibromatosis, psoriasis, vascular smooth cell proliferation associated with atherosclerosis, pulmonary fibrosis, arthritis, glomerulonephritis and post-surgical stenosis and restenosis.

The compounds of the invention may be useful in treatment of Alzheimer's disease, as suggested by the fact that cdk5 is involved in the phosphorylation of tau protein (J. Biochem. 117, 741-749, 1995).

The compounds of this invention, as modulators of apoptosis, may also be useful in the treatment of cancer, viral infections, prevention of AIDS development in HIV-infected individuals, autoimmune diseases and neurodegenerative disorders.

The compounds of this invention may be useful in inhibiting tumor angiogenesis and metastasis.

The compounds of the invention may also act as inhibitor of other protein kinases, e.g., protein kinase C in different isoforms, Met, PAK-4, PAK-5, ZC-1, STLK-2, DDR-2, Aurora 1, Aurora 2, Bub-1, PLK, Chk1, Chk2, HER2, raf1, MEK1, MAPK, EGF-R, PDGF-R, FGF-R, IGF-R, PI3K, weel kinase, Src, Abl, Akt, ILK, MK-2,IKK-2 Cdc7, Nek, and thus be effective in the treatment of diseases associated with other protein kinases.

The compounds of the invention are also useful in the treatment and prevention of radiotherapy-induced or chemotherapy-induced alopecia.

Accordingly, the present invention provides a method for treating cell proliferative disorders caused by and/or associated with an altered cell cycle dependent kinase activity, by administering to a mammal in need thereof an effective amount of a phenylacetamido-pyrazole derivative represented by formula (I):

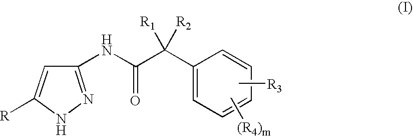

wherein

R is an optionally substituted C₃-C₅ cycloalkyl group;

R₁ and R₂, the same or different, represent hydrogen, halogen, amino, hydroxy or a group selected from straight or branched C₁-C₅ alkyl optionally substituted by amino or hydroxy, straight or branched C₁-C₅ perfluorinated alkyl or straight or branched C₁-C₅ alkoxy or, taken together with the carbon atom to which they are bonded, R₁ and R₂ form an exomethylene (>C═CH₂) group or a C₃-C₄ cycloalkyl group;

R₃, in position 3 or 4 of the phenyl ring, is a group of formula

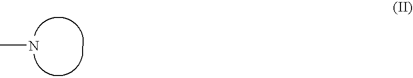

representing a 5 or 6 membered saturated or unsaturated nitrogen containing heterocycle; optionally containing from 1 to 2 additional heteroatoms, the same or different, selected from nitrogen, oxygen or sulfur; optionally substituted in any of the free positions by one or more groups selected from halogen; hydroxy; aminocarbonyl; C₁-C₄ alkylaminocarbonyl; oxo groups (>C═O, >S═O, >SO₂); exomethylene (>C═CH₂); straight or branched C₁-C₄ alkyl, perfluorinated alkyl, hydroxyalkyl or alkoxy groups; C₂-C₄ alkenyl or aryl groups; optionally condensed with carbocyclic or heterocyclic rings, either saturated or unsaturated, either monocyclic or bicyclic, each of which being optionally further substituted as above defined;

m is 0 or an integer from 1 to 4;

if present, each R₄ is, the same or different, halogen, hydroxy or a group selected from straight or branched C₁-C₄ alkyl, perfluorinated alkyl or alkoxy;

or a pharmaceutically acceptable salt thereof,

provided that:

a) when R is cyclopropyl and R₁ and R₂ are both hydrogen atoms, then the nitrogen containing heterocycle of formula (II) is other than 1-pyrrolidinyl, 2-oxo-1-pyrrolidinyl or 1,2,3-triazol-1-yl; and

b) when R is cyclopropyl, one of R₁ and R₂ is a hydrogen atom and the other is methyl, ethyl, n-propyl or n-butyl, then the nitrogen-containing heterocycle of formula (II) is other than 1,3-dihydro-2H-isoindol-2-yl or 1-oxo-1,3-dihydro-2H-isoindol-2-yl.

In a preferred embodiment of the method described above, the cell proliferative disorder is selected from the group consisting of cancer, Alzheimer's disease, viral infections, auto-immune diseases and neurodegenerative disorders.

Specific types of cancer that may be treated include, for instance, carcinoma, squamous cell carcinoma, hematopoietic tumors of myeloid or lymphoid lineage, tumors of mesenchymal origin, tumors of the central and peripheral nervous system, melanoma, seminoma, teratocarcinoma, osteosarcoma, xeroderma pigmentosum, keratoxanthoma, thyroid follicular cancer, and Kaposi's sarcoma.

In another preferred embodiment of the method described above, the cell proliferative disorder is selected from the group consisting of benign prostate hyperplasia, familial adenomatosis polyposis, neuro-fibromatosis, psoriasis, vascular smooth cell proliferation associated with atherosclerosis, pulmonary fibrosis, arthritis, glomerulonephritis and post-surgical stenosis and restenosis.

In addition, the inventive method provides tumor angiogenesis and metastasis inhibition. The inventive method may also provide cell cycle inhibition or cdk/cyclin dependent inhibition.

In addition to the above, the method object of the present invention provides treatment and prevention of radiotherapy-induced or chemotherapy-induced alopecia.

The present invention also provides a phenylacetamido-pyrazole derivative of formula

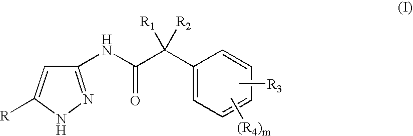

R is an optionally substituted C₃-C₅ cycloalkyl group;

R₁ and R₂, the same or different, represent hydrogen, halogen, amino, hydroxy or a group selected from straight or branched C₁-C₅ alkyl optionally substituted by amino or hydroxy, straight or branched C₁-C₅ perfluorinated alkyl or straight or branched C₁-C₅ alkoxy or, taken together with the carbon atom to which they are bonded, R₁ and R₂ form an exomethylene (>C═CH₂) group or a C₃-C₄ cycloalkyl group;

R₃, in position 3 or 4 of the phenyl ring, is a group of formula

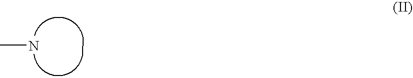

representing a 5 or 6 membered saturated or unsaturated nitrogen containing heterocycle; optionally containing from 1 to 2 additional heteroatoms, the same or different, selected from nitrogen, oxygen or sulfur; optionally substituted in any of the free positions by one or more groups selected from halogen; hydroxy; aminocarbonyl; C₁-C₄ alkylaminocarbonyl; oxo groups (>C═O, >S═O, >SO₂); exomethylene (>C═CH₂); straight or branched C₁-C₄ alkyl, perfluorinated alkyl, hydroxyalkyl or alkoxy groups; C₂-C₄ alkenyl or aryl groups; optionally condensed with carbocyclic or heterocyclic rings, either saturated or unsaturated, either monocyclic or bicyclic, each of which being optionally further substituted as above defined;

m is 0 or an integer from 1 to 4;

if present, each R₄ is, the same or different, halogen, hydroxy or a group selected from straight or branched C₁-C₄ alkyl, perfluorinated alkyl or alkoxy;

or a pharmaceutically acceptable salt thereof;

provided that:

a) when R is cyclopropyl and R₁ and R₂ are both hydrogen atoms, then the nitrogen containing heterocycle of formula (II) is other than 1-pyrrolidinyl, 2-oxo-1-pyrrolidinyl or 1,2,3-triazol-1-yl; and

b) when R is cyclopropyl, one of R₁ and R₂ is a hydrogen atom and the other is methyl, ethyl, n-propyl or n-butyl, then the nitrogen-containing heterocycle of formula (II) is other than 1,3-dihydro-2H-isoindol-2-yl or 1-oxo-1,3-dihydro-2H-isoindol-2-yl.

The present invention also includes methods of synthesizing the phenylacetamidopyrazole derivatives of formula (I), as well as the pharmaceutical compositions thereof. A more complete appreciation of the invention and many of the attendant advantages thereof will be readily obtained as the same becomes better understood by reference to the following detailed description.

### DETAILED DESCRIPTION OF THE INVENTION

Several amido-pyrazole derivatives are known in the art, for instance as pesticides, herbicides or even as therapeutic agents. Among them are, as an example, heteroaryl-pyrazoles active as p38 kinase inhibitors (WO 98/52941, G. D. Searle and Co.) and 3-amino-pyrazoles active as protein kinase inhibitors (WO 96/14843, COR Therapeutics, Inc.).

A class of amido-pyrazole and derivatives thereof, endowed with cyclin dependent kinase inhibitory activity, are also disclosed in U.S. Pat. No. 6,218,418 (corresponding to WO 01/12189) and WO 01/12188,both in the name of Pharmacia & Upjohn S.p.A and Pharmacia & Upjohn Co., which are herewith incorporated by reference.

The compounds of the present invention fall within the scope of the general formula of U.S. Pat. No. 6,218,418 but are not specifically exemplified therein.

In addition, a class of amido-pyrazoles containing a chromane moiety, active as cdk inhibitors, are also disclosed in co-pending, still unpublished, U.S. patent application Ser. No. 09/769,441,in the name of Pharmacia & Upjohn S.p.A, herewith incorporated by reference.

As it will be readily appreciated, the unsubstituted pyrazole ring nitrogens in the compounds of the invention are known to rapidly equilibrate, in solution, as admixtures of both tautomers:

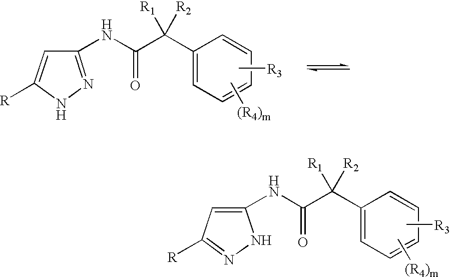

Accordingly, in the present invention and unless otherwise specified, where only one tautomer is indicated for the compounds of formula (I), the other is also within the scope of the present invention.

The compounds of formula (I) may have asymmetric carbon atoms and may therefore exist either as racemic admixtures or as individual optical isomers which are all within the scope of the present invention.

Just as an example, the compounds o f formula (I) wherein R₁ is alkyl, for instance methyl, and R₂ is hydrogen, have an asymmetric carbon atom and, hence, both the (R) and (S) optical isomers as well as the racemic (R,S) admixture are within the scope of the invention.

Likewise, the use as an antitumor agent of all the possible isomers and their admixtures and of both the metabolites and the pharmaceutically acceptable bio-precursors (otherwise referred to as pro-drugs) of the compounds of formula (I) are also within the scope of the present invention.

As used herein, unless otherwise specified, the term C₃-C₅ cycloalkyl comprises cyclopropyl, cyclobutyl and cyclopentyl.

With the term halogen atom, unless otherwise indicated, we intend iodine, bromine, chlorine or fluorine.

As used herein, unless otherwise indicated, the terms straight or branched C₁-C₅ alkyl or alkoxy groups include, for instance, methyl, ethyl, n-propyl, isopropyl, n-butyl, isobutyl, sec-butyl, tert-butyl, n-pentyl, isopentyl, methoxy, ethoxy, n-propoxy, isopropoxy, n-butoxy, isobutoxy, sec-butoxy, tert-butoxy and the like.

With the term straight or branched perfluorinated C₁-C₅ alkyl group we intend any of the above alkyl groups which are substituted by more than one fluorine atom such as, for instance, trifluoromethyl, trifluoroethyl and the like.

As used herein, unless otherwise indicated, the term C₂-C₄ alkenyl includes, for instance, a group selected from vinyl, allyl, 1-propenyl, isopropenyl, 1-butenyl, 2-butenyl, 3-butenyl and the like.

The term aryl includes carbocyclic or heterocyclic hydrocarbons, with from 1 to 2 ring moieties either fused or linked to each other by single bonds, wherein at least one of the rings is aromatic.

Examples of aryl groups are, for instance, phenyl, biphenyl, α- or β-naphthyl, dihydronaphthyl, thienyl, benzothienyl, furyl, benzofuranyl, pyrrolyl, imidazolyl, pyrazolyl, thiazolyl, isothiazolyl, oxazolyl, isoxazolyl, pyridyl, pyrazinyl, pyrimidinyl, pyridazinyl, indolyl, isoindolyl, purinyl, quinolyl, isoquinolyl, dihydroquinolinyl, quinoxalinyl, benzodioxolyl, indanyl, indenyl, triazolyl, tetrazolyl and the like.

Unless otherwise specified, the term heterocycle, which also encompasses aromatic heterocycles also referred to as aryl or heteroaryl groups, includes a 5 or 6 membered saturated or unsaturated carbocycle, wherein one or more carbon atoms are replaced by one or more heteroatoms selected from nitrogen, oxygen and sulphur.

When referring to the group R₃ represented by formula (II), the 5 or 6 membered nitrogen-containing heterocycle is bonded in position 3 or 4, that is meta or para, of the phenyl ring in formula (I), through the nitrogen atom.

Unless otherwise specified, the said nitrogen-containing heterocycle is saturated, partly unsaturated or fully unsaturated.

Examples of the above nitrogen-containing heterocycles of formula (II) are, for instance, pyrrolidine, pyrroline, pyrrole, imidazolidine, imidazoline, imidazole, pyrazolidine, pyrazoline, pyrazole, piperidine, piperazine, morpholine, triazole, triazolidine, oxazole, oxazoline, oxazolidine, pyrimidine, isothiazolidine and the like.

As formerly indicated, the above ring structures of formula (II) may be further condensed, through any one of the available bonds, with saturated or unsaturated, either monocyclic or bicyclic rings such as, for instance, cyclohexane, cyclopentane, cyclohexene, cyclopentene, bicyclo[2.2.1]heptane, bicyclo[2.2.1]hept-2-ene, bicyclo[2.2.2]octane, bicyclo[2.2.2]oct-2-ene, benzene, naphthalene, pyridine, pyrimidine, pyrazine, tetrahydrofuran, dihydrofuran, tetrahydropyridazine and the like.

In addition, the said heterocycles of formula (II) may be optionally further substituted, by one or more of the aforementioned groups, in any of their free positions.

As an example, the heterocycles of formula (II) may be substituted by oxo groups so as to give rise to pyrrolidinone, piperidinone, imidazolidinone, oxazolidinone rings and the like; one or two oxo groups may be also bonded to a sulphur atom so as to yield, for instance, thiazolidine-1,1-oxide, isothiazolidine-1,1-oxide and the like.

According to the above indicated substituents and unless otherwise specified, any of the above R or R₃ groups may be optionally substituted in any of the free positions by one or more groups independently selected from halogen, nitro, oxo groups, cyano, alkyl, perfluorinated alkyl, hydroxyalkyl, aryl, arylalkyl, heterocyclyl, heterocyclylalkyl, cycloalkyl, hydroxy, alkoxy, perfluorinated alkoxy, aryloxy, heterocyclyloxy, methylenedioxy, alkylcarbonyloxy, arylcarbonyloxy, carboxy, alkoxycarbonyl, aryloxycarbonyl, cycloalkyloxycarbonyl, amino, ureido, alkylamino, dialkylamino, arylamino, diarylamino, formylamino, alkylcarbonylamino, arylcarbonylamino, heterocyclylcarbonylamino, alkoxycarbonylamino, alkoxyimino, alkylsulfonylamino, arylsulfonylamino, formyl, alkylcarbonyl, arylcarbonyl, cycloalkylcarbonyl, heterocyclylcarbonyl, aminocarbonyl, alkylaminocarbonyl, dialkylaminocarbonyl, alkylsulfonyl, arylsulfonyl, aminosulfonyl, alkylaminosulfonyl, dialkylaminosulfonyl, arylthio and alkylthio.

Unless otherwise indicated, any of the terms such as, for instance, alkylthio, alkylamino, dialkylamino, alkoxycarbonyl, alkoxycarbonylamino, heterocyclylcarbonyl, heterocyclylcarbonylamino, cycloalkyloxycarbonyl and the like, include groups wherein the alkyl, alkoxy, aryl, cycloalkyl and heterocyclyl moieties are as above defined.

When referring to the compounds of formula (I) wherein the nitrogen-containing heterocycle of formula (II) may give rise to equivalent tautomeric form, for instance as reported below

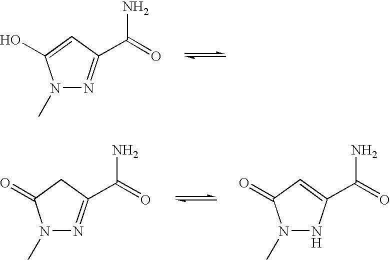

it is clear to the man skilled in the art that all of the said forms are to be intended as within the scope of the present invention.

Pharmaceutically acceptable salts of the compounds of formula (I) include the acid addition salts with inorganic or organic acids, e.g., nitric, hydrochloric, hydrobromic, sulphuric, perchloric, phosphoric, acetic, trifluoroacetic, propionic, glycolic, lactic, oxalic, malonic, malic, maleic, tartaric, citric, benzoic, cinnamic, mandelic, methanesulphonic, isethionic and salicylic acid, as well as the salts with inorganic or organic bases, e.g., alkali or alkaline-earth metals, especially sodium, potassium, calcium or magnesium hydroxides, carbonates or bicarbonates, acyclic or cyclic amines, preferably methylamine, ethylamine, diethylamine, triethyl amine or piperidine.

Preferred compounds of the invention are the compounds of formula (I) wherein R is a C₃-C₅ cycloalkyl group; one of R₁ and R₂ is hydrogen and the other is a halogen atom or a straight or branched C₁-C₄ alkyl, perfluorinated alkyl or aminoalkyl group; and R₃, R₄ and m have the above reported meanings.

Also preferred are the compounds of formula (I) wherein R is a C₃-C₅ cycloalkyl group; R₁ and R₂ are both halogen atoms, preferably fluorine, or taken together with the carbon atom to which they are bonded form an exomethylene group (>C═CH₂) or a C₃-C₄ cycloalkyl group; and R₃, R₄ and m have the above reported meanings.

Even more preferred compounds of formula (I), within the above two classes, are the compounds wherein R, R₁, R₂, R₄ and m are as above defined and R₃, being optionally further substituted and/or condensed as above defined, is selected from the group consisting of:

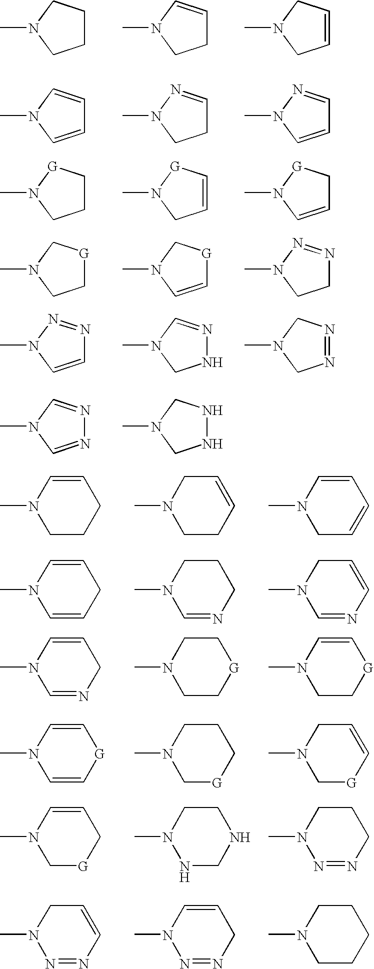

wherein G represents a group —NH—, —O—, —S— or —SO₂—.

Still more preferred are the compounds of formula (I) wherein R, R₁, R₂, R₄ and m are as above defined and R₃ is a group selected from: 1-pyrrolidinyl; 2-oxo-1-pyrrolidinyl; 3-methyl-2-oxo-1-pyrrolidinyl; 2-methyl-5-oxo-1-pyrrolidinyl; 2-ethyl-5-oxo-1-pyrrolidinyl; 2-oxo-5-phenyl-1-pyrrolidinyl; 2-oxo-1,3-oxazolidin-3-yl; 2-oxo-3,3a,6,6a-tetrahydrocyclopenta[b]pyrrol-1(2H)-yl; 2-(hydroxymethyl)-5-oxo-1-pyrrolidinyl; 3-hydroxy-2-oxo-1-pyrrolidinyl; 4-hydroxy-2-oxo-1-pyrrolidinyl; 3-hydroxy-4,4-dimethyl-2-oxo-1-pyrrolidinyl; 2-oxo-3-pyrrolidinecarboxamide; 5-oxo-3-pyrrolidinecarboxamide; 5-oxo-2-pyrrolidinecarboxamide; 1,3-dioxo-1,3-dihydro-2H-isoindol-2-yl; 1-hydroxy-3-oxo-1,3-dihydro-2H-isoindol-2-yl; 1-oxooctahydro-2H-isoindol-2-yl; 2,5-dioxo-2,5-dihydro-1H-pyrrol-1-yl; 2,5-dioxo-1-pyrrolidinyl; 2,5dioxo-1-pyrrolidinyl; 6-methoxy-1-oxo-1,3-dihydro-2H-isoindol-2-yl; 1,1-dioxido-3-oxo-1,2-benzisothiazol-2(3H)-yl; 1,3-dioxo-1,3-dihydro-2H-benzo[f]isoindol-2-yl; 1-oxo-1H-benzo[de]isoquinolin-2(3H)-yl; 1H-pyrrol-1-yl; 7-hydroxy-3H-[1,2,3]triazolo[3H-[1,2,3]triazolo[4,5-d]pyrimidin-3-yl; 1-oxo-1,3-dihydro-2H-isoindol-2-yl; 2,4-dioxotetrahydro-1(2H)-pyrimidinyl; 3,5-dioxo-1,2,4-triazolidin-4-yl; 2,5-dioxo-1-imidazolidinyl; 2,4-dioxo-1-imidazolidinyl; 2-oxo-1-imidazolidinyl; 2-oxo-2,3-dihydro-1H-imidazol-1-yl; 2-oxo-2,3-dihydro-1H-imidazol-1-yl; 5-oxo-1,5-dihydro-4H-1,2,4-triazol-4-yl; 5-oxo-1,5-dihydro-4H-1,2,4-triazol-4-yl; 5-hydroxy-1H-pyrazole-3-carboxamide; 3-oxo-1-pyrazolidinyl; 3,5-dioxo-1-pyrazolidinyl; 2,4-dioxo-3,4-dihydro-1(2H)-pyrimidinyl; 3,5-dioxo-4-morpholinyl; 2-oxo-1-piperidinyl; 1-piperazinyl; 4-morpholinyl and 1-piperidinyl.

Another class of preferred compounds of the invention are the derivatives of formula (I) wherein R is a C₃-C₅ cycloalkyl group, R₁ is a hydrogen atom or a methyl group, R₂ is a hydrogen atom, m is 0 and R₃, in position 4 of the phenyl ring (para), is a 2-oxo-1,3-oxazolidin-3-yl group; these latter derivatives may be thus conveniently represented according to formula (I′) below:

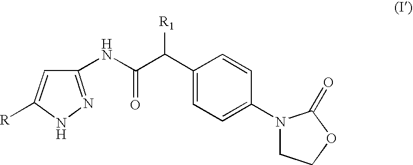

Still more preferred, in this latter class of derivatives of formula (I′), are the compounds wherein R is a cyclopropyl group.

Also preferred are the above compounds of formula (I′) wherein R₁ is a methyl group.

Even more preferred are the compounds wherein the above features are combined together so as to get a compound of formula (I′) wherein R is cyclopropyl and R₁ is methyl.

Even more preferred is the compound of formula (I′) wherein R is cyclopropyl and R₁ is methyl, in its (S) optical form, namely the compound (2S)-N-(5-cyclopropyl-1H-pyrazol-3-yl)-2-[4-(2-oxo-1,3-oxazolidin-3-yl)phenyl]propanamide.

Examples of preferred compounds of the invention of formula (I), also comprehensive of the above derivatives of formula (I′), optionally in the form of pharmaceutically acceptable salts, include:

1. N-(5-cyclopropyl-1H-pyrazol-3-yl)-2-[4-(2-oxo-1,3-oxazolidin-3-yl)phenyl]propanamide;

2. (2R)-N-(5-cyclopropyl-1H-pyrazol-3-yl)-2-[4-(2-oxo-1,3-oxazolidin-3-yl)phenyl]propanamide;

3. (2S)-N-(5-cyclopropyl-1H-pyrazol-3-yl)-2-[4-(2-oxo-1,3-oxazolidin-3-yl)phenyl]propanamide;

4. N-(5-cyclopropyl-1H-pyrazol-3-yl)-2-[4-(2-oxo-1,3-oxazolidin-3-yl)phenyl]acetamide;

5. N-(5-cyclobutyl-1H-pyrazol-3-yl)-2-[4-(2-oxo-1,3-oxazolidin-3-yl)phenyl]propanamide;

6. (2R)-N-(5-cyclobutyl-1H-pyrazol-3-yl)-2-[4-(2-oxo-1,3-oxazolidin-3-yl)phenyl]propanamide;

7. (2S)-N-(5-cyclobutyl-1H-pyrazol-3-yl)-2-[4-(2-oxo-1,3-oxazolidin-3-yl)phenyl]propanamide;

8. N-(5-cyclobutyl-1H-pyrazol-3-yl)-2-[4-(2-oxo-1,3-oxazolidin-3-yl)phenyl]acetamide;

9. N-(5-cyclopentyl-1H-pyrazol-3-yl)-2-[4-(2-oxo-1,3-oxazolidin-3-yl)phenyl]propanamide;

10. (2R)-N-(5-cyclopentyl-1H-pyrazol-3-yl)-2-[4-(2-oxo-1,3-oxazolidin-3-yl)phenyl]propanamide;

11. (2S)-N-(5-cyclopenyl-1H-pyrazol-3-yl)-2-[4-(2-oxo-1,3-oxazolidin-3-yl)phenyl]propanamide;

12. N-(5-cyclopentyl-1H-pyrazol-3-yl)-2-[4-(2-oxo-1,3-oxazolidin-3-yl)phenyl]acetamide;

13. N-(5-cyclopropyl-1H-pyrazol-3-yl)-2-[4-(2-oxo-1-pyrrolidinyl)phenyl]propanamide;

14. (2S)-N-(5-cyclopropyl-1H-pyrazol-3-yl)-2-[4-(2-oxo-1-pyrrolidinyl)phenyl]propanamide;

15. (2R)-N-(5-cyclopropyl-1H-pyrazol-3-yl)-2-[4-(2-oxo-1-pyrrolidinyl)phenyl]propanamide;

16. N-(5-cyclobutyl-1H-pyrazol-3-yl)-2-[4-(2-oxo-1-pyrrolidinyl)phenyl]propanamide;

17. (2S)-N-(5-cyclobutyl-1H-pyrazol-3-yl)-2-[4-(2-oxo-1-pyrrolidinyl)phenyl]propanamide;

18. (2R)-N-(5-cyclobutyl-1H-pyrazol-3-yl)-2-[4-(2-oxo-1-pyrrolidinyl)phenyl]propanamide;

19. N-(5-cyclopropyl-1H-pyrazol-3-yl)-2-fluoro-2-[4-(2-oxo-1-pyrrolidinyl)phenyl]acetamide;

20. 2-chloro-N-(5-cyclopropyl-1H-pyrazol-3-yl)-2-[4-(2-oxo-1-pyrrolidinyl)phenyl]acetamide;

21. N-(5-cyclopropyl-1H-pyrazol-3-yl)-2,2-difluoro-2-[4-(2-oxo-1-pyrrolidinyl)phenyl]acetamide;

22. N-(5-cyclopropyl-1H-pyrazol-3-yl)-3,3,3-trifluoro-2-[4-(2-oxo-1-pyrrolidinyl)phenyl]acetamide;

23. 3-amino-N-(5-cyclopropyl-1H-pyrazol-3-yl)-2-[4-(2-oxo-1-pyrrolidinyl)phenyl]propanamide;

24. N-(5-cyclopropyl-1H-pyrazol-3-yl)-2-[4-(3-methyl-2-oxo-1-pyrrolidinyl)phenyl]acetamide;

25. N-(5-cyclopropyl-1H-pyrazol-3-yl)-2-[4-(3-methyl-2-oxo-1-pyrrolidinyl)phenyl]propanamide;

26. N-(5-cyclopropyl-1H-pyrazol-3-yl)-2-[4-(2-methyl-5-oxo-1-pyrrolidinyl)phenyl]acetamide;

27. N-(5-cyclopropyl-1H-pyrazol-3-yl)-2-[4-(2-methyl-5-oxo-1-pyrrolidinyl)phenyl]propanamide;

28. N-(5-cyclopropyl-1H-pyrazol-3-yl)-2-[4-(2-ethyl-5-oxo-1-pyrrolidinyl)phenyl]acetamide;

29. N-(5-cyclopropyl-1H-pyrazol-3-yl)-2-[4-(2-ethyl-5-oxo-1-pyrrolidinyl)phenyl]propanamide;

30. N-(5-cyclopropyl-1H-pyrazol-3-yl)-2-[4-(2-oxo-5-phenyl-1-pyrrolidinyl)phenyl]acetamide;

31. N-(5-cyclopropyl-1H-pyrazol-3-yl)-2-[4-(2-oxo-5-phenyl-1-pyrrolidinyl)phenyl]propanamide;

32. N-(5-cyclopropyl-1H-pyrazol-3-yl)-2-[4-(2-oxo-3,3a,6,6a-tetrahydrocyclopenta[b]pyrrol-1(2H)-yl)phenyl]acetamide;

33. N-(5-cyclopropyl-1H-pyrazol-3-yl)-2-[4-(2-oxo-3,3a,6,6a-tetrahydrocyclopenta[b]pyrrol-1(2H)-yl)phenyl]propanamide;

34. N-(5-cyclopropyl-1H-pyrazol-3-yl)-2-{4-[2-(hydroxymethyl)-5-oxo-1-pyrrolidiny]phenyl}acetamide;

35. N-(5-cyclopropyl-1H-pyrazol-3-yl)-2-{4-[2-(hydroxymethyl)-5-oxo-1-pyrrolidinyl]phenyl}propanamide;

36. N-(5-cyclopropyl-1H-pyrazol-3-yl)-2-[4-(3-hydroxy-2-oxo-1-pyrrolidinyl)phenyl]acetamide;

37. N-(5-cyclopropyl-1H-pyrazol-3-yl)-2-[4-(3-hydroxy-2-oxo-1-pyrrolidinyl)phenyl]propanamide;

38. N-(5-cyclopropyl-1H-pyrazol-3-yl)-2-[4-(4-hydroxy-2-oxo-1-pyrrolidinyl)phenyl]acetamide;

39. N-(5-cyclopropyl-1H-pyrazol-3-yl)-2-[4-(4-hydroxy-2-oxo-1-pyrrolidinyl)phenyl]propanamide;

40. N-(5-cyclopropyl-1H-pyrazol-3-yl)-2-[4-(3-hydroxy-4,4-dimethyl-2-oxo-1-pyrrolidinyl)phenyl]acetamide;

41. N-(5-cyclopropyl-1H-pyrazol-3-yl)-2-[4-(3-hydroxy-4,4-dimethyl-2-oxo-1-pyrrolidinyl)phenyl]propanamide;

42. 1-(4-{2-[(5-cyclopropyl-1H-pyrazol-3-yl)amino]-2-oxoethyl}phenyl)-2-oxo-3-pyrrolidinecarboxamide;

43. 1-(4-{2-[(5-cyclopropyl-1H-pyrazol-3-yl)amino]-1-methyl-2-oxoethyl}phenyl)-2-oxo-3-pyrrolidinecarboxamide;

44. 1-(4-{2-[(5-cyclopropyl-1H-pyrazol-3-yl)amino]-2-oxoethyl}phenyl)-5-oxo-3-pyrrolidinecarboxamide;

45. 1-(4-{2-[(5-cyclopropyl-1H-pyrazol-3-yl)amino]-1-methyl-2-oxoethyl}phenyl)-5-oxo-3-pyrrolidinecarboxamide;

46. 1-(4-{2-[(5-cyclopropyl-1H-pyrazol-3-yl)amino]-2-oxoethyl}phenyl)-5-oxo-2-pyrrolidinecarboxamide;

47. 1-(4-{2-[(5-cyclopropyl-1H-pyrazol-3-yl)amino]-1-methyl-2-oxoethyl}phenyl)-5-oxo-2-pyrrolidinecarboxamide;

48. N-(5-cyclopropyl-1H-pyrazol-3-yl)-2-[4-(1,3-dioxo-1,3-dihydro-2H-isoindol-2-yl)phenyl]acetamide;

49. N-(5-cyclopropyl-1H-pyrazol-3-yl)-2-[4-(1,3-dioxo-1,3-dihydro-2H-isoindol-2-yl)phenyl]propanamide;

50. N-(5-cyclopropyl-1H-pyrazol-3-yl)-2-[4-(1-hydroxy-3-oxo-1,3-dihydro-2H-isoindol-2-yl)phenyl]propanamide;

51. N-(5-cyclopropyl-1H-pyrazol-3-yl)-2-[4-(1-oxooctahydro-2H-isoindol-2-yl)phenyl]acetamide;

52. N-(5-cyclopropyl-1H-pyrazol-3-yl)-2-[4-(1-oxooctahydro-2H-isoindol-2-yl)phenyl]propanamide;

53. N-(5-cyclopropyl-1H-pyrazol-3-yl)-2-[4-(2,5-dioxo-2,5-dihydro-1H-pyrrol-1-yl)phenyl]acetamide;

54. N-(5-cyclopropyl-1H-pyrazol-3-yl)-2-[4-(2,5-dioxo-2,5-dihydro-1H-pyrrol-1-yl)phenyl]propanamide;

55. N-(5-cyclopropyl-1H-pyrazol-3-yl)-2-[4-(2,5-dioxo-1-pyrrolidinyl)phenyl]acetamide;

56. N-(5-cyclopropyl-1H-pyrazol-3-yl)-2-[4-(2,5-dioxo-1-pyrrolidinyl)phenyl]propanamide;

57. N-(5-cyclopropyl-1H-pyrazol-3-yl)-2-[4-(6-methoxy-1-oxo-1,3-dihydro-2H-isoindol-2-yl)phenyl]propanamide;

58. N-(5-cyclopropyl-1H-pyrazol-3-yl)-2-[4-(1,1-dioxido-3-oxo-1,2-benzisothiazol-2-(3H)-yl)phenyl]acetamide;

59. N-(5-cyclopropyl-1H-pyrazol-3-yl)-2-[4-(1,1-dioxido-3-oxo-1,2-benzisothiazol-2(3H)-yl)phenyl]propanamide;

60. N-(5-cyclopropyl-1H-pyrazol-3-yl)-2-[4-(1,3-dioxo-1,3-dihydro-2H-benzo[f]isoindol-2-yl)phenyl]propanamide;

61. N-(5-cyclopropyl-1H-pyrazol-3-yl)-2-[4-(1-oxo-1H-benzo[de]isoquinolin-2(3H)-yl)phenyl]propanamide;

62. N-(5-cyclopropyl-1H-pyrazol-3-yl)-2-[4-(1H-pyrrol-1-yl)phenyl]acetamide;

63. N-(5-cyclopropyl-1H-pyrazol-3-yl)-2-[4-(1H-pyrrol-1-yl)phenyl]propanamide;

64. N-(5-cyclopropyl-1H-pyrazol-3-yl)-2-[4-(7-hydroxy-3H-[1,2,3]triazolo[4,5-d]pyrimidin-3-yl)phenyl]acetamide;

65. N-(5-cyclopropyl-1H-pyrazol-3-yl)-2-[4-(3H-[1,2,3]triazolo[4,5-d]pyrimidin-3-yl)phenyl]propanamide;

66. 2-[2-chloro-4-(1-oxo-1,3-dihydro-2H-isoindol-2-yl)phenyl]-N-(5-cyclopropyl-1H-pyrazol-3-yl)acetamide;

67. 2-[2-chloro-4-(1-oxo-1,3-dihydro-2H-isoindol-2-yl)phenyl]-N-(5-cyclopropyl-1H-pyrazol-3-yl)propanamide;

68. N-(5-cyclopropyl-1H-pyrazol-3-yl)-2-[4-(2,4-dioxotetrahydro-1(2H)-pyrimidinyl)phenyl]acetamide;

69. N-(5-cyclopropyl-1H-pyrazol-3-yl)-2-[4-(2,4-dioxotetrahydro-1-(2H)-pyrimidinyl)phenyl]propanamide;

70. N-(5-cyclopropyl-1H-pyrazol-3-yl)-2-[4-(3,5-dioxo-1,2,4-triazolidin-4-yl)phenyl]acetamide;

71. N-(5-cyclopropyl-1H-pyrazol-3-yl)-2-[4-(3,5-dioxo-1,2,4-triazolidin-4-yl)phenyl]propanamide;

72. N-(5-cyclopropyl-1H-pyrazol-3-yl)-2-[4-(2,5-dioxo-1-imidazolidinyl)phenyl]acetamide;

73. N-(5-cyclopropyl-1H-pyrazol-3-yl)-2-[4-(2,5-dioxo 1-imidazolidinyl)phenyl]propanamide;

74. N-(5-cyclopropyl-1H-pyrazol-3-yl)-2-[4-(2,4-dioxo-1-imidazolidinyl)phenyl]acetamide;

75. N-(5-cyclopropyl-1H-pyrazol-3-yl)-2-[4-(2,4-dioxo-1-imidazolidinyl)phenyl]propanamide;

76. N-(5-cyclopropyl-1H-pyrazol-3-yl)-2-[4-(2-oxo-1-imidazolidinyl)phenyl]acetamide;

77. N-(5-cyclopropyl-1H-pyrazol-3-yl)-2-[4-(2-oxo-1-imidazolidinyl)phenyl]propanamide;

78. N-(5-cyclopropyl-1H-pyrazol-3-yl)-2-[4-(2-oxo-2,3-dihydro-1H-imidazol-1-yl)phenyl]acetamide;

79. N-(5-cyclopropyl-1H-pyrazol-3-yl)-2-[4-(2-oxo-2,3-dihydro-1H-imidazol-1-yl)phenyl]propanamide;

80. N-(5-cyclopropyl-1H-pyrazol-3-yl)-2-[4-(5-oxo-1,5-dihydro-4H-1,2,4-triazol-4-yl)phenyl]propanamide;

81. N-(5-cyclopropyl-1H-pyrazol-3-yl)-2-[4-(5-oxo-1,5-dihydro-4H-1,2,4-triazol-4-yl)phenyl]propanamide;

82. 1-(4-{2-[(5-cyclopropyl-1H-pyrazol-3-yl)amino]-2-oxoethyl}phenyl)-5-hydroxy-1H-pyrazol-3-carboxamide;

83. 1-(4-{2-[(5-cyclopropyl-1H-pyrazol-3-yl)amino]-1-methyl-2-oxoethyl}phenyl)-5-hydroxy-1H-pyrazole-3-carboxamide;

84. N-(5-cyclopropyl-1H-pyrazol-3-yl)-2-[4-(3-oxo-1-pyrazolidinyl)phenyl]acetamide;

85. N-(5-cyclopropyl-1H-pyrazol-3-yl)-2-[4-(3-oxo-1-pyrazolidinyl)phenyl]propanamide;

86. N-(5-cyclopropyl-1H-pyrazol-3-yl)-2-[4-(3,5-dioxo-1-pyrazolidinyl)phenyl]acetamide;

87. N-(5-cyclopropyl-1H-pyrazol-3-yl)-2-[4-(3,5-dioxo-1-pyrazolidinyl)phenyl]propanamide;

88. N-(5-cyclopropyl-1H-pyrazol-3-yl)-2-[4-(2,4-dioxo-3,4-dihydro-1(2H)-pyrimidinyl)phenyl]acetamide;

89. N-(5-cyclopropyl-1H-pyrazol-3-yl)-2-[4-(2,4-dioxo-3,4-dihydro-1(2H)-pyrimidinyl)phenyl]acetamide;

90. N-(5-cyclopropyl-1H-pyrazol-3-yl)-2-[4-(3,5-dioxo-4-morpholinyl)phenyl]acetamide;

91. N-(5-cyclopropyl-1H-pyrazol-3-yl)-2-[4-(3,5-dioxo-4-morpholinyl)phenyl]propanamide;

92. N-(5-cyclopropyl-1H-pyrazol-3-yl)-2-[4-(2-oxo-1-piperidinyl)phenyl]acetamide;

93. N-(5-cyclopropyl-1H-pyrazol-3-yl)-2-[4-(2-oxo-1-piperidinyl)phenyl]propanamide;

94. N-(5-cyclopropyl-1H-pyrazol-3-yl)-2-[4-(1-piperazinyl)phenyl]acetamide;

95. N-(5-cyclopropyl-1H-pyrazol-3-yl)-2-[4-(1-piperazinyl)phenyl]propanamide;

96. N-(5-cyclopropyl-1H-pyrazol-3-yl)-2-[4-(4-morpholinyl)phenyl]acetamide;

97. N-(5-cyclopropyl-1H-pyrazol-3-yl)-2-[4-(4-morpholinyl)phenyl]propanamide;

98. N-(5-cyclopropyl-1H-pyrazol-3-yl)-2-[4-(1-piperidinyl)phenyl]acetamide;

99. N-(5-cyclopropyl-1H-pyrazol-3-yl)-2-[4-(1-pyrrolidinyl)phenyl]propanamide;

100. N-(5-cyclopropyl-1H-pyrazol-3-yl)-2-[4-(1-piperidinyl)phenyl]propanamide.

As formerly indicated, a further object of the invention is represented by the process for preparing the compounds of formula (I), hence including also those of formula (I′).

The compounds of formula (I) and the salts thereof, object of the present invention, may be thus obtained by a process comprising:

a) reacting the compounds of formula (III) or the regioisomers of formula (IIIa)

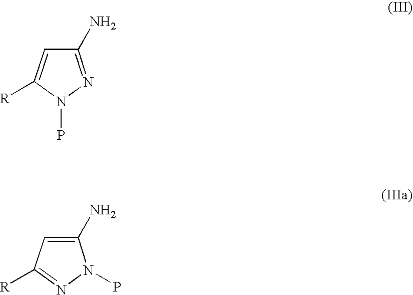

wherein R is as above defined and P represents a suitable nitrogen-pyrazole protecting group, with the compounds of formula (IV)

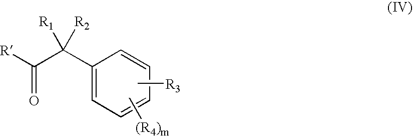

wherein R₁, R₂, R₃, R₄ and m have the above reported meanings and R′ represents hydroxy or a halogen atom, thus obtaining the compounds of formula (V) or (Va)

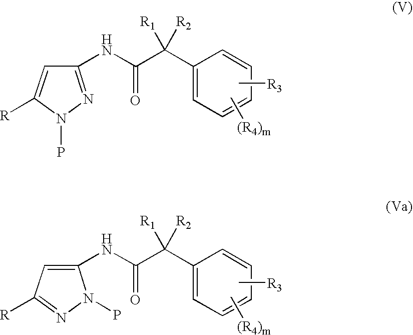

b) and deprotecting the compounds of formula (V) or (Va) so as to obtain the derivatives of formula (I) and, if desired, converting them into pharmaceutically acceptable salts thereof.

The above process is an analogy process which can be carried out according to well known methods.

According to step a) of the process, the reaction of the compounds of formula (III) or (IIIa) with the compounds of formula (IV) can be carried out in the presence of a coupling agent, for instance a carbodiimide such as 1,3-dicyclohexylcarbodiimide, 1,3-diisopropylcarbodiimide or 1-(3-dimethylaminopropyl)-3-ethylcarbodiimide, optionally in the presence of a tertiary base such as triethylamine, N-methylmorpholine, N,N-diisopropylethylamine or pyridine.

The reaction occurs in a suitable solvent such as, for example, dichloromethane, chloroform, tetrahydrofuran, diethylether, 1,4-dioxane, acetonitrile, toluene or N,N-dimethylformamide, at a temperature ranging from about −10° C. to reflux for a suitable time, for instance from about 30 minutes to about 96 hours.

Alternatively, the reaction of the compounds of formula (III) or (IIIa) with the compounds of formula (IV) can be also carried out, for example, by a mixed anhydride method, that is by using an alkyl chloroformate such as ethyl, isobutyl or isopropylchloroformate in the presence of a tertiary base such as triethylamine, N-methylmorpholine, N,N-diisopropylethylamine or pyridine, in a suitable solvent such as toluene, dichloromethane, chloroform, tetrahydrofuran, acetonitrile, diethylether, 1,4-dioxane or N,N-dimethylformamide, at a temperature ranging from about −30° C. to room temperature.

When starting from the compounds of formula (IV) wherein R′ is a halogen atom, preferably chlorine, the reaction can be carried out in the presence of a base such as triethylamine, N-methylmorpholine, N,N-diisopropylethylamine or pyridine, in a suitable solvent such as dichloromethane, chloroform, diethylether, tetrahydrofuran, 1,4-dioxane, acetonitrile, toluene or N,N-dimethylformamide, at a temperature ranging from about 0° C. to reflux.

As far as the compounds of formula (III) or (IIIa) are concerned, suitable P groups are those conventionally used to protect pyrazole-nitrogen atoms. Preferably, for both compounds (III) and (IIIa), P represents a tert-butoxycarbonyl (boc) group.

In step b) of the process, the compounds of formula (V) or (Va) are converted into the desired derivatives of formula (I) by deprotecting the pyrazole-nitrogen atom, according to conventional methods.

As an example, deprotection from “boc” may occurr by acidic hydrolysis, for instance in the presence of trifluoroacetic, formic or sulphuric acid, in a suitable solvent such as dichloromethane, 1,4-dioxane or ethanol, at a temperature ranging from about 0° C. to room temperature.

The compounds of formula (III) or (IIIa) are known or may be prepared according to known methods by starting from the corresponding deprotected derivatives of formula

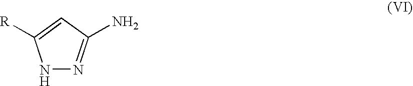

wherein R is as above defined.

For a reference to the preparation of the compounds of formula (III) see, for instance, the aforementioned U.S. Pat. No. 6,218,418.

When preparing the compounds of formula (IIIa), the compounds of formula (VI) are protected, for instance as “boc” derivatives through reaction with tert-butoxycarbonylanhydride, in the presence of a dichloromethane/water admixture and of an inorganic base such as sodium hydroxide, carbonate or hydrogenocarbonate. This same reaction may be also carried out in dichloromethane, chloroform, toluene, tetrahydrofuran or 1,4-dioxane and in the presence of an organic base, for instance triethylamine or N,N-diisopropylethylamine, at a temperature ranging from about 0° C. to room temperature.

Alternatively, the compounds of formula (IIIa), for instance protected as “boc” derivatives, can be prepared by reacting the corresponding keto-nitrile of formula

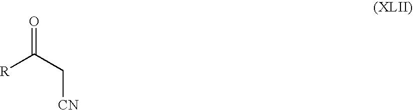

wherein R is as described above, with tert-butylcarbazate in a suitable solvent such as ethanol or methanol, in the presence of a base such as triethylamine or N,N-diisopropylethylamine, at a temperature ranging from about 0° C. to room temperature.

Also the compounds of formula (IV) are known or may be prepared according to known methods. As an example, the compounds of formula (IV) wherein R′ is a halogen atom, for instance chlorine, are prepared from the corresponding derivatives of 736 formula (IV) wherein R′ is hydroxy, by reacting these latter derivatives with oxalyl chloride or thionyl chloride, according to conventional methods for preparing acyl halides. The reaction may occurr in the presence of a suitable solvent, for instance dichloromethane, tetrahydrofuran, ethylacetate or toluene, at temperature ranging from room temperature to reflux.

The compounds of formula (IV) wherein R′ is hydroxy and R₁, R₂, R₃, R₄ and m have the above reported meanings may be prepared by a process comprising the reaction of a derivative of formula

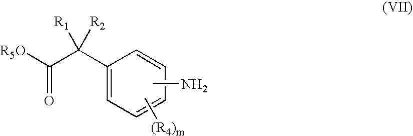

wherein R₅ is an alkyl group and the amino group is in position 3 or 4 of the phenyl ring, with a suitable reagent, as reported in details below, allowing the formation of a compound of formula

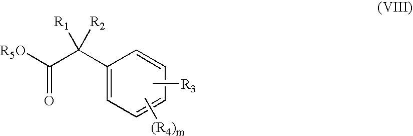

bearing the desired R₃ residue, followed by the acidic or basic hydrolysis of the ester group.

The above hydrolysis to yield the compounds of formula (IV) can be carried out with a base such as sodium or potassium hydroxide, in a suitable solvent such as methanol or ethanol or, alternatively under acidic conditions, in the presence of hydrochloric, hydrobromic or sulphuric acid, in a suitable solvent such as acetic acid, tetrahydrofuran or 1,4-dioxane, at a temperature ranging from room temperature to reflux.

The compounds of formula (VIII) are known or can be prepared according to known methods.

As an example, the compounds of formula (VIII) wherein R₃ is a pyrrolidine or piperidine ring, either saturated or unsaturated, as per formula (IX) below wherein the dotted lines represent a single or double bond, R₁, R₂, R₄, R₅ and m are as above defined, Z is methylene, ethylene, C═O or CH—OH, R₆ and R₇ are, each independently, hydrogen atoms or methyl, ethyl, phenyl, hydroxymethyl, hydroxy, aminocarbonyl or allyl groups or, taken together, may form a keto group (═O) or a fused aryl or cycloalkyl group optionally further substituted

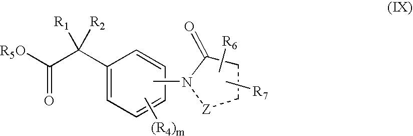

can be prepared by reacting the compounds of formula (VII) with the compounds of formula (X)

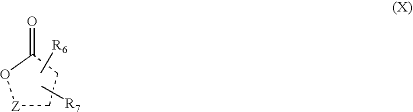

wherein Z, R₆ and R₇ are as described above, in the presence of an acid such as acetic or hydrochloric acid, at a temperature ranging from about 80° C. to about 200° C.

The compounds of formula (VIII) wherein R₃ is a triazolidinedione ring system, as per formula (XI) below wherein R₁, R₂, R₄, R₅ and m are as above defined

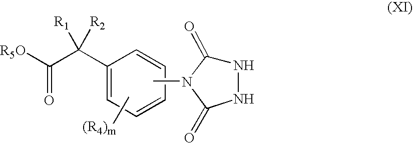

can be prepared by reacting the compounds of formula (VII) with bi-urea, in a suitable solvent such as N-methylpyrrolidone, N,N-dimethylformamide or dimethylsulphoxide, at a temperature ranging from room temperature to reflux.

The compounds of formula (VIII) wherein R₃ is a pyrrolidine, piperidine, piperazine or morpholine system, as per formula (XII) below wherein R₁, R₂, R₄, R₅ and m are as above defined, n is 1 or 2 and Y is CH₂, NH or O

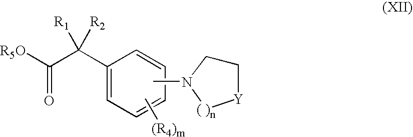

can be prepared by reacting the compounds of formula (VII) with the compounds of formula (XIII)

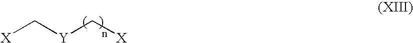

wherein Y and n are as bove defined and X is a halogen atom or hydroxy.

The above reaction with the compounds of formula (XIII) wherein X is halogen can be carried out in a suitable solvent such as methanol, acetonitrile, 1,4-dioxane or toluene, in the presence of a base such as sodium hydrogenocarbonate or carbonate or triethylamine, at a temperature ranging from room temperature to reflux. Alternatively, by using the compounds of formula (XIII) wherein X is hydroxy, the reaction can be carried out in the presence of a catalyst, for instance a mixture of rutenium chloride and triphenylphosphine, in a suitable solvent such as dichloromethane, tetrahydrofuran or 1,4-dioxane, at a temperature ranging from room temperature to reflux.

The compounds of formula (VI) wherein R₃ is a 1,3-oxazolidin-2-one system, as per formula (XIV) below wherein R₁, R₂, R₄, R₅ and m are as above defined

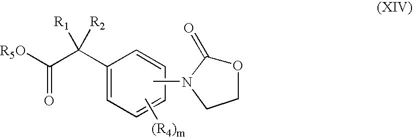

can be prepared by reacting the compounds of formula (VII) with 2-chloroethylchloroformate under Schotten-Bauman conditions.

More in particular, the reaction can be carried out in the presence of aqueous sodium hydroxide and toluene at room temperature, or with a conventional organic base under anhydrous conditions; the intermediate compound thus prepared, is then treated with an aqueous base, for instance s odium hydroxide or methoxide, in a suitable solvent such as water or water-methanol admixtures, at a temperature ranging from room temperature to about 60° C.

The compounds of formula (VIII) wherein R₃ is a 3,5-morpholinedione system, as per formula (XV) below wherein R₁, R₂, R₄, R₅ and m are as above defined

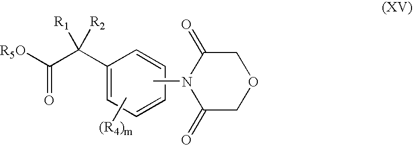

can be prepared by reacting the compounds of formula (VII) with diglycolic acid, in the presence of a suitable solvent, for instance water-tetrahydrofliran admixtures, at refluxing temperature.

The compounds of formula (VII) wherein R₃ is a 2,4-dihydro-3H-1,2,4-triazol-3-one system, as per formula (XVI) below wherein R₁, R₂, R₄, R₅ and m are as above defined

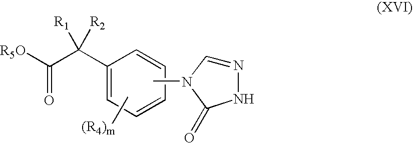

can be prepared by reacting the compounds of formula (VII) with methylhydrazino carboxylate in the presence of p-toluensulfonic acid and trimethylorthoformate, in a suitable solvent such as methanol at a temperature of about 50-60° C., and by subsequently adding sodium methoxide under refluxing temperature for a time ranging from about 2 to about 6 hours.

The compounds of formula (VIII) wherein R₃ is a 2-imidazolidinone system, as per formula (XVII) below wherein R₁, R₂, R₄, R₅ and m are as above defined

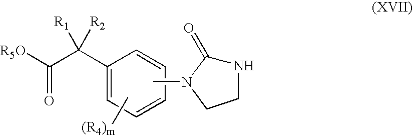

can be prepared by reacting the compounds of formula (VII) with 2-chloroethylisocyanate and, subsequently, with a base, or with 2-hydroxyethylisocyanate and, subsequently, with p-toluensulfonyl chloride and a base.

When using 2-chloroethylisocyanate, the reaction can be carried out in a suitable solvent such as dichloromethane, chloroform, tetrahydrofuran, acetonitrile or 1,4-dioxane, thus affording an intermediate compound which is treated with a base such as sodium or potassium hydroxide in a suitable solvent, for instance tetrahydrofuran, 1,4-dioxane or N,N-dimethylformamide, at a temperature ranging from room temperature to about 60° C.

Alternatively, when using 2-hydroxyethylisocyanate, the raction may occur in a suitable solvent such as dichloromethane, chloroform, tetrahydrofuiran, acetonitrile or 1,4-dioxane, thus affording an intermediate compound that is treated with p-toluensulfonylchloride in the presence of a base such as potassium tert-butoxide, in a suitable solvent such as diethylether, tetrahydrofuran or 1,4-dioxane, at a temperature ranging from room temperature to reflux.

The compounds of formula (VII) wherein R₃ is a 1,3-dihydro-2H-imidazol-2-one, as per formula (XVIII) wherein R₁, R₂, R₄, R₅ and m are as above defined

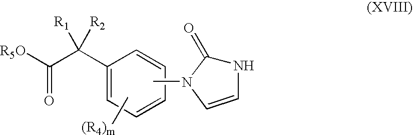

can be prepared through a process comprising reacting the compounds of formula (VII) with a carbonyl equivalent that is, for instance, phosgene, diphosgene, triphosgene, carbonyldiimidazole or disuccinimidocarbonate, thus obtaining the compounds of formula (XIX) below

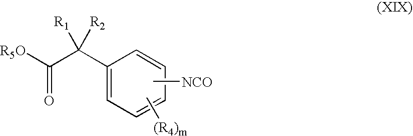

and by reacting the compounds of formula (XIX) with 2-aminoacetaldeyde dimethylacetale followed by acidic treatment.

The reaction of the compounds of formula (VII) with the carbonyl equivalent is carried out in a suitable solvent such as diethylether, tetrahydrofuran, 1,4-dioxane or N,N-dimethylformamide, at a temperature ranging from room temperature to about 80° C.

The subsequent reaction with 2-aminoacetaldehyde dimethylacetale can be carried out in a suitable solvent such as dichloromethane, chloroform, tetrahydrofuran or 1,4-dioxane, at room temperature, thus affording an intermediate compound which is directly treated with an acid, for instance a water:trifluoroacetic acid=1:1 admixture, at a temperature ranging from room temperature to reflux.

The compounds of formula (VEII) wherein R₃ is a 2,4-imidazolidindione system, as per formula (XX) wherein R₁, R₂, R₄, R₅ and m are as above defined

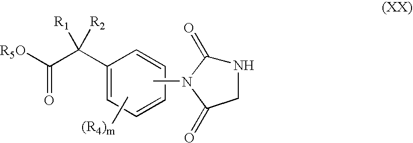

can be prepared by reacting the compounds of formula (VII) with ethyl isocyanatoacetate, in a suitable solvent such as ethanol or methanol, thus affording an intermediate compound which is directly treated with an acid, for instance hydrochloric acid, in a suitable solvent such as a ethanol-water admixture, at a temperature ranging from about 40° C. to reflux.

The compounds of formula (VEII) wherein R₃ is a 2,4-dihydro-3H-1,2,4-triazol-3-one system, as per formula (XXI) below wherein R₁, R₂, R₄, R₅ and m are as above defined

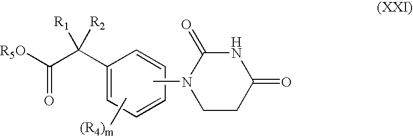

can be prepared by a process comprising reacting the compounds of formula (VII) with acrylic acid, thus obtaining the compounds of formula (XXII)

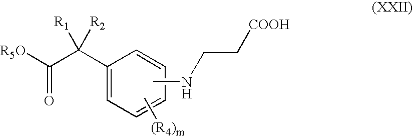

and by reacting the compounds of formula (XXII) with urea.

The reaction with acrylic acid can be carried out in a suitable solvent such as dichloromethane, acetonitrile, 1,4-dioxane or N,N-dimethylformamide, at a temperature ranging from room temperature to reflux; the subsequent reaction with urea can be carried out in the presence of an acid such as hydrochloric, sulphuric or phosphoric acid, at refluxing temperature.

The compounds of formula (VIII) wherein R₃ is a 2,4(1H,3H)-pyrimidinedione system, as per formula (XXIII) below wherein R₁, R₂, R₄, R₅ and m are as above defined

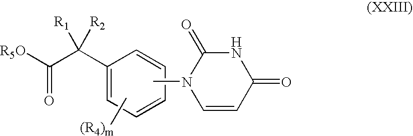

can be prepared by reacting the compounds of formula (VII) with ethyl (2E)-3-ethoxy-2-propenoylcarbamate, prepared as described in Journal of American Chemical Society 111, 374 (1989), in the presence of a suitable solvent such as 1-butanol and at refluxing temperature and, subsequently, by treatment with aqueous sodium hydroxide.

The compounds of formula (VIII) wherein R₃ is a 2,4-dihydro-3H-pyrazol-3-one or a 2,5-pyrazolidindione, as per formula (XXIV) below wherein R₁, R₂, R₄, R₅ and m are as above defined and W is CH₂ or C═O

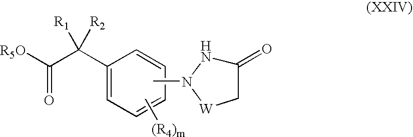

can be prepared by a process comprising reacting the compounds of formula (VII) with sodium nitrite in acidic medium and by treating the resulting diazonium salt with sodium sulphite or tin chloride, thus obtaining the compounds of formula (XXV)

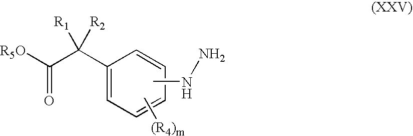

and by reacting the compounds of formula (XXV) with 3-chloropropionic acid or malonic acid, thus obtaining the compounds of formula (XXIV) wherein W is CH₂ or CO, respectively.

The reaction of the compounds of formula (VII) with sodium nitrite is carried out in the presence of an acid, for instance aqueous hydrochloric acid, at about 0° C.; the subsequent treatment with a reducing a gent such as sodium sulphite in water or tin trichloride in aqueous hydrochloric acid, allows to obtain the compounds of formula (XXV).

The reaction of the compounds of formula (XXV) with 3-chloropropionic acid, to yield the compounds of formula (XXIV) wherein W is CH₂ can be carried out without a solvent, at a temperature ranging from about 150° C. to about 210° C., for a time ranging from about 2 to about 8 hours. The reaction of the compounds of formula (XXV) with malonic acid to yield the compounds of formula (XXIV) wherein W is CO, instead, can be carried out in the presence of phosphorus oxychloride in a suitable solvent such as dichloromethane or chloroform, at a temperature ranging from about 20° C. to about 100° C.

The compounds of formula (VIII) wherein R₃ is a pyrazole system substituted by an aminocarbonyl group as described in formula (XXVI) below wherein R₁, R₂, R₄, R₅ and m are as above defined

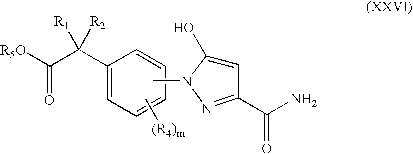

can be prepared by a process comprising reacting the compounds of formula (VII) with sodium nitrite, in acidic medium, and then by treating the resulting diazonium salt with diethylacetylsuccinate, thus obtaining the compounds of formula (XXVII)

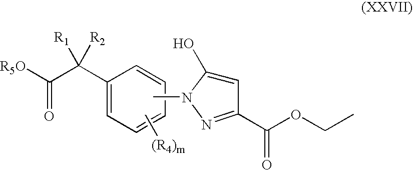

and by reacting the compounds of formula (XXVII) under ammonolysis conditions. The reaction of the compounds of formula (VII) with sodium nitrite is carried out as formerly indicated, in aqueous hydrochloric acid at about 0° C. The subsequent treatment of the diazonium salt with diethylacetylsuccinate is carried out in the presence of a base such as sodium hydrogenocarbonate or carbonate, in water, at a temperature ranging from room temperature to about 60° C.

The reaction of the compounds of formula (XXVII) under ammonolysis conditions occurrs in the presence of gaseous ammonia, in methanol or ethanol, or with 30% ammonium hydroxide solution, in a suitable solvent such as methanol, ethanol or N,N-dimethylformamide. at a temperature ranging from about −10° C. to room temperature.

The compounds of formula (IX) wherein Z is CH₂, R₆ is —CONH₂ in position 3 of the 2-pyrrolidinone system and R₇ is hydrogen, as per formula (XXVIII) below wherein R₁, R₂, R₄, R₅ and m are as above defined

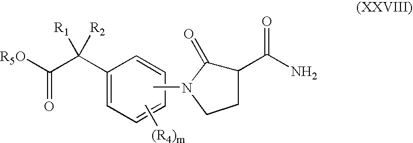

can be prepared by a process comprising reacting the compounds of formula (VII) with 1,1-cyclopropanedicarboxylic acid or with 6,6-dimethyl-5,7-dioxaspiro[2.5]octane-4,8-dione, thus obtaining the compounds of formula (XXIX)

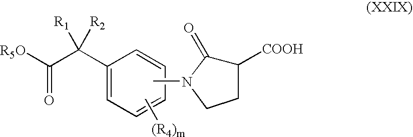

and by reacting them with ammonia in the presence of an alkylchloroformate or with 1-hydroxybenzotriazole ammonium salt in the presence of a carbodiimide.

The reaction of the compounds of formula (VII) with 1,1-cyclopropanedicarboxylic acid to yield the compounds of formula (XXIX) can be carried out in water or in water-ethanol, water-tetrahydrofuran or water-acetonitrile admixtures, at a temperature ranging from about 40° C. to about 80° C. Alternatively, the same reaction can be carried out with 6.6-dimethyl-5,7-dioxaspiro[2.5]octane-4,8-dione in a suitable solvent such as toluene or xylene at refluxing temperature.

The reaction of the compounds of formula (XXIX) with ammonia can be carried out in the presence of an alkylchloroformate as a condensing agent, for instance ethylchloroformate, in a suitable solvent such as dichloromethane, chloroform, acetonitrile, tetrahydrofuran, 1,4-dioxane or N,N-dimethylformamide, at a temperature ranging from about −10° C. to room temperature.

Alternatively this same reaction can be carried out with 1-hydroxybenzotriazole ammonium salt in the presence of a carbodiimide as a condensing agent, for instance 1,3-dicyclohexylcarbodiimide, 1,3-diisopropylcarbodiimide or 1-(3-dimethylaminopropyl)-3-ethylcarbodiimide, in a suitable solvent such as dichloromethane, chloroform, tetrahydrofyran, acetonitrile, 1,4-dioxane or N,N-dimethylformamide, optionally in the presence of a base such as triethylamine, N-methylmorpholine or N,N-diisopropylethylamine, at a temperature ranging from about −20° C. to room temperature.

The compounds of formula (IX) wherein Z is CH₂, R₆ is —CONH₂ in position 4 of the 2-pyrrolidinone system and R₇ is hydrogen, as per formula (XXX) below wherein R₁, R₂, R₄, R₅ and m are as above defined

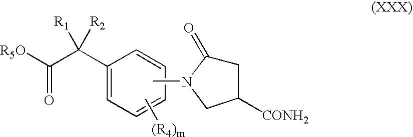

can be prepared by a process comprising reacting the compounds of formula (VII) with itaconic acid, thus obtaining the compounds of formula (XXXI)

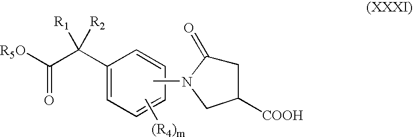

and by converting the carboxy group into carboxamide, according to conventional techniques for preparing amides.

The reaction of the compounds of formula (VII) with itaconic acid to yield the compounds of formula (XXXI) can be optionally carried out in the presence of a suitable solvent such as ethanol or methanol, optionally in the presence of a suitable catalyst such as p-toluensulphonic acid, at a temperature ranging from about 60° C. to about 100° C.

The conversion of the carboxylic acid derivative of formula (XXXI) to the corresponding amide (XXX) can be carried out by working as formerly described for preparing the compounds of formula (XXVIII) from those of formula (XXIX).

The compounds of formula (VIII) wherein R₃ is a 2,4-imidazolidindione system as described in formula (XXXII) wherein R₁, R₂, R₄, R₅ and m are as above defined

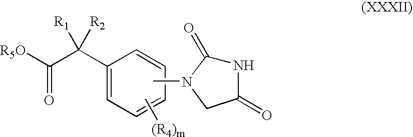

can be prepared by a process comprising reacting the compounds of formula (VII) with potassium cyanate, thus obtaining the compounds of formula (XXXIII)

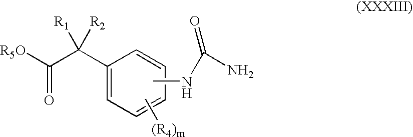

and by reacting the compounds of formula (XXXIII) with ethyl 2-chloroacetate. The reaction with potassium cyanate can be carried out in a suitable solvent such as acetonitrile, tetrahydrofuran, 1,4-dioxane or N,N-dimethylformamide, at a temperature ranging from room temperature to about 70° C.

The subsequent reaction of the compounds of formula (XXXIII) with ethyl 2-chloroacetate can be carried out in the presence of a base such as sodium or potassium hydroxide in a suitable solvent such as N,N-dimethylformamide, tetrahydrofuran or diethylether, at a temperature ranging from room temperature to about 60° C.

The compounds of formula (VIII) wherein one of R₁ or R₂ is hydrogen and the other is alkyl (e.g. R₁ as hydrogen and R₂ as akyl), and R₃, R₄, R₅ and m are as described above, can be prepared by reacting the compounds of formula (VIII) wherein R₁ and R₂ are both hydrogen atoms, with the compounds of formula (XXXIV) below

R₂-Hal (XXXIV)

wherein R₂ is alkyl and Hal is a halogen atom, by working in the presence of a base such as potassium tert-butoxide or sodium hydoxide, in a suitable solvent such as tetrahydrofuran, 1,4-dioxane or N,N-dimethylformamide, at a temperature ranging from about −70° C. to room temperature.

The compounds of formula (VIII) wherein one of R₁ or R₂ is hydrogen or fluorine and the other is fluorine, and R₃, R₄, R₅ and m are as described above, can be prepared by reacting the compounds of formula (VIII) wherein R₁ and R₂ are both hydrogen atoms with N-fluorosaccharinsultam.

This reaction can be carried out in the presence of a base such as lithium or potassium hexamethyldisilazide or lithium diisopropylamide, in a suitable solvent such as diethylether, tetrahydrofuran or 1,4-dioxane, at a temperature ranging from about −78° C. to about 0° C.

The compounds of formula (VIII) wherein one of R₁ or R₂ is hydrogen or chlorine and the other is chlorine, and R₃, R₄, R₅ and m are as described above, can be prepared by reacting the compounds of formula (VIII) wherein R₁ and R₂ are hydrogen atoms with chlorine and a catalyst such as phosphor tribromide, or with N-chlorosuccinimide and hydrochloric acid.

The reaction with chlorine can be carried out in a suitable solvent such as carbon tetrachloride, at a temperature ranging from room temperature to about 60° C. Alternatively, when using N-chlorosuccinimide and hydrochloric acid, the reaction can be carried out in a suitable solvent such as 1,4-dioxane, tetrahydrofurane or carbon tetrachloride, at a temperature ranging from room temperature to reflux.

The compounds of formula (VIII) wherein one of R₁ or R₂ is a minomethyl and the other is hydrogen, and R₃, R₄, R₅ and m are as described above, can be prepared by reacting the compounds of formula (VIII) wherein R₁ and R₂ are both hydrogen atoms with formaldehyde and ammonia under Mannich conditions.

The compounds of formula (VIII) wherein one of R₁ or R₂ is trifluoromethyl and the other is hydrogen, and R₃, R₄ and R₅ are as described above, can be prepared by reacting the compounds of formula (VIII) wherein R₁ and R₂ are hydrogen atoms, with S-(trifluoromethyl)dibenzothiophenium trifluoromethanesulfonate, in the presence of a base such as sodium or potassium hydroxide and B-phenylcatecholborane as a catalyst, in a suitable solvent such as diethyleher, tetrahydroffiran or 1,4-dioxane, at a temperature ranging from about 0° C. to room temperature.

In addition to the above, herewith provided is also a novel process for preparing the compounds of the invention of formula (I′)

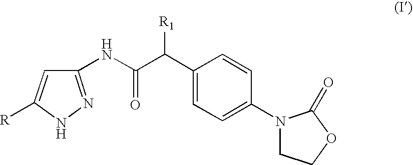

wherein R is a C₃-C₅ cycloalkyl group, R₁ is a hydrogen atom or a methyl group, and the pharmaceutically acceptable salts thereof; which process comprises:

a) reacting the compounds of formula (XXXV)

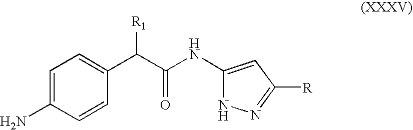

wherein R and R₁ are as described above, with chloroethylchloroformate in the presence of a base such as pyridine, triethylamine or N,N-diisopropylethylamine and in a suitable solvent such as pyridine, dichloromethane, tetrahydrofuran or N,N′-dimethylformamide, at a temperature ranging from about −20° C. to room temperature, thus obtaining the compounds of formula (XXXVI)

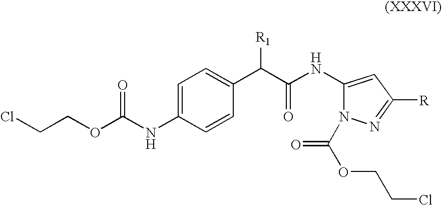

wherein R and R₁ are as described above;

b) reacting the compounds of formula (XXXVI) with a suitable base such as potassium carbonate or 1,8-diazabicyclo[5.4.0]undec-7-ene in a suitable solvent such as N,N-dimethylformamide or N,N-dimethylacetamide, at a temperature ranging from room temperature to reflux, thus obtaining the compounds of formula (XXXVII)

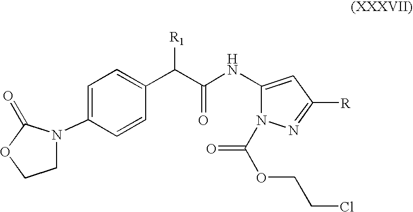

c) and by hydrolyzing the compounds of formula (XXXVII) with a base such as triethylamine or potassium carbonate in a suitable solvent such as methanol.

In their turn, the compounds of formula (XXXV) can be prepared by a process comprising:

a) reacting the compounds of formula (XXXVIII)

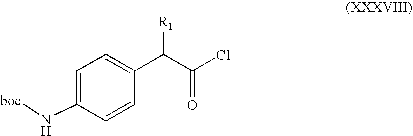

wherein boc stands for tert-butoxycarbonyl and R₁ is as described above, with the compounds of formula (IIIa) wherein R is as described above, thus obtaining the compounds of formula (XXXIX)

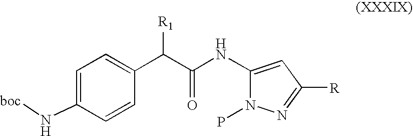

wherein P is a suitable nitrogen pyrazole protecting group, for instance boc;

b) and by hydrolyzing the compounds of formula (XXXIX) in acidic medium.

The reaction between the compounds of formula (XXXVIII) and the compounds of formula (IIIa) can be carried out in the presence of a conventional organic base such as triethylamine, N-methylmorpholine or N,N-diisopropylethylamine, in a suitable solvent such as chloroform, dichloromethane, tetrahydrofuran or 1,4-dioxane, at a temperature ranging from about 0° C. to room temperature.

The hydrolysis of the compounds of formula (XXXIX) to afford the compounds of formula (XXXV) can be carried out in a suitable solvent such as dichloromethane or ethanol with a suitable acid such as trifluoroacetic, formic or sulphuric acid at room temperature.

The compounds of formula (XXXIII) can be prepared by a process comprising:

a) reacting the compound of formula (XL) wherein R₁ is as above described

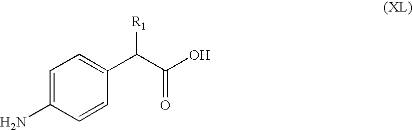

with tert-butoxycarbonylanhydride, thus obtaining the compounds of formula (XLI)

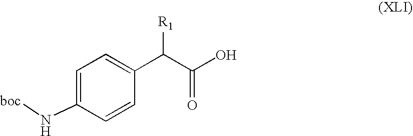

b) and by reacting the compounds of formula (XLI) with oxalyl or thionyl chloride.

The reaction between the compounds of formula (XL) with tertbutoxycarbonylanhydride can be carried out in a suitable solvent such as water/1,4-dioxane or water/dichloromethane admixtures, in the presence of a base such as sodium carbonate or sodium hydroxide, at a temperature ranging from about 0° C. to room temperature.

The reaction between the compounds of formula (XLI) with oxalyl or thionyl chloride can be carried out in a suitable solvent such as dichloromethane, tetrahydrofuran or ethyl acetate, in the presence of a catalytic amount of dimethylformamide at a temperature ranging from about 0° C. to reflux.

As it will be readily appreciated, if the compounds of formula (I) also comprising those of formula (I′), prepared according to the processes described above, are obtained as an admixture of isomers, their separation into the single isomers of formula (I), or of formula (I′), according to conventional techniques, is within the scope of the present invention.

Conventional techniques for racemate resolution include, for instance, partitioned crystallization of diastereoisomeric salt derivatives or preparative chiral HPLC.

Also, the optional conversion of a compound of formula (I) or (I′) into another compound of formula (I) or (I′), its optional salification or, on the other hand, the conversion of a salt into the free compound, can be all carried out according to known methods.

When preparing the compounds of formula (I) or (I′), optional functional groups within both the starting materials or the intermediates thereof which could give rise to unwanted side reactions are preferably protected according to conventional techniques.

Likewise, the conversion of these protected compounds into the free deprotected derivatives may be carried out according to well known methods.

Unless otherwise noted, any of the reagents being used in the above processes as well as any of the compounds of formula (VI), (VII), (X), (XIII), (XXXIV), (XL) and (XLII), together with any other additional starting material being used in the above processes, are all known or can be easily prepared according to well-known methods.

### Pharmacology

The compounds of formula (I) are active as cdk/cyclin ihibitors as they gave positive results when tested according to the following procedure.

The inhibiting activity of putative cdk/cyclin inhibitors and the potency of selected compounds was determined through a method of assay based on the use of the MultiScreen-PH 96 well plate (Millipore), in which phosphocellulose filter paper was placed at each well bottom allowing binding of positive charged substrate after a washing/filtration step.

When a radioactivity labelled phosphate moiety was transferred by the ser/threo kinase to the filter-bound histone, light emitted was measured in a scintillation counter.

### The Inhibition Assay of cdk2/Cyclin A Activity was Performed According to the Following Protocol

Kinase reaction: 1.5 μM histone H1 substrate, 25 μM ATP (0.5 uCi P33g-ATP), 100 ng Cyclin A/cdk2 complex, 10 μM inhibitor in a final volume of 100 μl buffer (TRIS HCl 10 mM pH 7.5, MgCl2 10 mM, 7.5 mM DTT) were added to each well of a 96 U bottom well plate. After 10 min at 37° C. incubation, reaction was stopped by 20 μl EDTA 120 mM.

Capture: 100 μl were transferred from each well MultiScreen plate, to allow substrate binding phosphocellulose filter. Plates were then washed 3 times with 150 μl/well PBS Ca++/Mg++ free and filtered by MultiScreen filtration system.

Detections: filters were allowed to dry at 37° C., then 100 μl/well scintillant were added and 33P labelled histone H1 was detected by radioactivity counting in the Top-Count instrument.

Results: data were analyzed and expressed as % inhibition referred to total activity of enzyme (=100%).

All compounds showing inhibition >50% were further analyzed in order to study and define the kinetic-profile of the inhibitor via Ki calculation.

The protocol used was the same described above, except for ATP and substrate concentrations. Either the concentrate of ATP and histone H1 substrate were varied: 4, 8, 12, 24, 48 μM for ATP (containing proportionally diluted P33g-ATP) and 0.4, 0.8, 1.2, 2.4, 4.8 μM for histone were used in absence and presence of two different, properly chosen inhibitor concentrations.

Experimental data were analyzed by the computer program “SigmaPlot” for Ki determination, using a random bireactant system equation:

$$v = \frac{ {V}_{max} \begin{matrix}( A ) ( B ) \\ aKAKB\end{matrix}}{1 + ( A ) + ( B ) + ( A ) ( B ) \\ KA KB aKAKB}$$

where A=ATP and B=histone H1.

In addition, the inhibiting activity of putative cdk/cyclin inhibitors and the potency of selected compounds was determined using a method of assay based on the use of a SPA (Scintillation Proximity Assay) 96 well plate assay. The assay is based on the ability of streptavidin-coated SPA beads to capture a biotinylated peptide derived from a phosphorylation site of histone.

When a radioactivity labelled phosphate moiety was transferred by the ser/threo kinase to the biotinylated histone peptide, light emitted was measured in scintillation counter.

### The Inhibition Assay of cdk5/p25 Activity was Performed According to the Following Protocol

Kinase reaction: 1.0 μM biotinylated histone peptide substrate, 0.25 uCi P33g-ATP, 4 nM cdk2/p25 complex, 0-100 μM] inhibitor in a final volume of 100 μl buffer (Hepes 20 mM pH 7.5, MgCl2 15 mM, 1 mM DTT) were added to each well of a 96 U bottom well plate. After 20 min at 37° C. incubation, the reaction was stopped by the addition of 500 ug SPA beads in phosphate-buffered saline containing 0.1% Triton X-100, 50 M ATP and 5 mM EDTA. The beads were allowed to settle, and the radioactivity incorporated in the 33P-labelled peptide was detected in a Top Count scintillation counter.

Results: Data were analyzed and expressed as % Inhibition using the formula:

100×(1-(Unknown−Bkgd)/(Enz. Control−Bkgd))

IC50 values were calculated using a variation of the four parameter logistics equation:

Y=100/[1+10\hat{ }{(Log EC50−X)*Slope}]

Where X=log(uM) and Y=% Inhibition.

Given the above inhibition assays, the compounds of formula (I) of the invention resulted to possess a remarkable cdk inhibitory activity. These compounds are thus useful in therapy against proliferative disorders caused by and/or associated with an altered cell cycle dependent kinase activity.

In particular, when tested against cdk2/A, a representative compound of the invention, namely (2S)-N-(5-cyclopropyl-1H-pyrazol-3-yl)-2-[4-(2-oxo-1,3-oxazolidin-3-yl)phenyl]propanamide, resulted to possess an inhibitory activity, expressed as IC₅₀, of 8 nM.

By restricting the unregulated proliferation of tumor cells, the compounds of formula (I) are therefore useful in therapy in the treatment of various tumors such as, for instance, carcinomas, e.g., mammary carcinoma, carcinoma, bladder carcinoma, colon carcinoma, ovary endometrial tumors, sarcomas, e.g., soft tissue and bone sarcomas, and the hematological malignancies such as, e.g., leukemias.

In addition, the compounds of formula (I) are also useful in the treatment of other cell proliferative disorders such as psoriasis, vascular smooth cell proliferation a ssocited with atherosclerosis and post-surgical stenosis a restenosis, and in the treatment of Alzheimer's disease.

The compounds of the present invention can be administered either as single agents or, alternatively, in combination with known anticancer treatments such as radiation therapy or chemotherapy regimen in combination with cytostatic or cytotoxic agents, antibiotic-type agents, alkylating agents, antimetabolite agents, hormonal agents, immunological agents, interferon-type agents, cyclooxygenase inhibitors (e.g. COX-2 inhibitors), matrixmetalloprotease inhibitors, telomerase inhibitors, tyrosine kinase inhibitors, anti-growth factor receptor agents, anti-HER agents, anti-EGFR agents, anti-angiogenesis agents, famesyl transferase inhibitors, ras-raf signal transduction pathway inhibitors, cell cycle inhibitors, other cdks inhibitors, tubulin binding agents, topoisomerase I inhibitors, topoisomerase II inhibitors, and the like.

As an example, the compounds of the invention can be administered in combination with one or more chemotherapeutic agents such as, for instance, exemestane, formestane, anastrozole, letrozole, fadrozole, taxane derivatives, e.g. paclitaxel and docetaxel, encapsulated taxanes, camptothecin derivatives, e.g. CPT-11 and SN-38, anthracycline glycosides, e.g. doxorubicin, idarubicin, epirubicin, etoposide, navelbine, vinblastine, carboplatin, cisplatin, estramustine phosphate and derivatives thereof, celecoxib, tamoxifen, raloxifen, Sugen SU-5416,Sugen SU-6668, Herceptin, and the like, optionally within liposomal formulations thereof.

If formulated as a fixed dose, said combination products employ the compounds of this invention within the dosage range described below and the other pharmaceutically active agent within the approved dosage range.

Compounds of formula (I) may be used sequentially with known anticancer agents when a combination formulation is inappropriate.

The compounds of formula (I) of the present invention, suitable for administration to a mammal, e.g., to humans, can be administered by the usual routes and the dosage, level depends upon the age, weight, conditions of patient and the administration route. For example, a suitable dosage adopted for oral administration of a compound of formula (I) may range from about 10 to about 500 mg per dose, from 1 to 5 times daily. The compounds of the invention can be administered in a variety of dosage forms, e.g., orally, in the form tablets, capsules, sugar or film coated tablets, liquid solutions or suspensions; rectally in the form suppositories; parenterally, e.g., intramuscularly, or intravenous and/or intrathecal and/or intraspinal injection or infusion.

The present invention also includes pharmaceutical compositions comprising a compound of formula (I) or a pharmaceutically acceptable salt thereof in association with a pharmaceutically acceptable excipient, which may be a carrier or a diluent.

The pharmaceutical compositions containing the compounds of the invention are usually prepared following convention methods and are administered in a pharmaceutically suitable form.

For example, the solid oral forms may contain, together with the active compound, diluents, e.g., lactose, dextrose saccharose, sucrose, cellulose, corn starch or potato starch; lubricants, e.g., silica, talc, stearic acid, magnesium or calcium stearate, and/or polyethylene glycols; binding agents, e.g., starches, arabic gum, gelatin methylcellulose, carboxymethylcellulose or polyvinyl pyrrolidone; disintegrating agents, e.g., a starch, alginic acid, alginates or sodium starch glycolate; effervescing mixtures; dyestuffs; sweeteners; wetting agents such as lecithin, polysorbates, laurylsulphates; and, in general, non-toxic and pharmacologically inactive substances used in pharmaceutical formulations. These pharmaceutical preparations may be manufactured in known manner, for example, by means of mixing, granulating, tabletting, sugar-coating, or film-coating processes.

The liquid dispersions for oral administration may be, e.g, syrups, emulsions and suspensions.

The syrups may contain as carrier, for example, saccharose or saccharose with glycerine and/or mannitol and sorbitol.

The suspensions and the emulsions may contain as carrier, for example, a natural gum, agar, sodium alginate, pectin, methylcellulose, carboxymethylcellulose, or polyvinyl alcohol.

The suspension or solutions for intramuscular injections may contain, together with the active compound, a pharmaceutically acceptable carrier, e.g., sterile water, olive oil, ethyl oleate, glycols, e.g., propylene glycol, and, if desired, a suitable amount of lidocaine hydrochloride.

The solutions for intravenous injections or infusions may contain as a carrier, for example, sterile water or preferably they may be in the form of sterile, aqueous isotonic saline solutions or they may contain as a carrier propylene glycol,

The suppositories may contain together with the active compound a pharmaceutically acceptable carrier, e.g., cocoa butter, polyethylene glycol, a polyoxyethylene sorbitan fatty acid ester surfactant or lecithin.

With the aim of better illustrate the present invention, without posing any limitation to it, the following examples are now given.

### EXAMPLE 1

### Tert-Butyl-5-amino-3-cyclopropyl-1H-pvrazole-1-carboxylate

(Method A) 0.81 g (6.6 mmol) of 3-cyclopropyl-1H-pyrazole-5-amine were dissolved in 20 ml of 2M sodium hydrate and 20 ml of dichloromethane. 2.8 g (13.2 mmol) of tert-butoxycarbonylanhydride were added and the mixture was maintained at room temperature under stirring overnight. The organic layer was separated, washed with water, dried over anhydrous sodium sulphate and evaporated. The title compound was crystallised from n-hexane (1 g, 71% yield).

ESI (+) MS: m/z 224 (100, MH+); 1H-NMR (400 MHz, DMSO-d6) ppm: 6.29 (s, 1H, H-pyrazole), 1.95 (m, 1H, CH-cyclopropyl), 1.46 (s, 9H, tert-butyl), 1.35-1.28 (2m, 4H, CH2-cyclopropyl).

According to the same method, but employing 3-cyclobutyl- or 3-cyclopentyl-1H-pyrazole-5-amine, the following compounds were prepared:

tertbutyl-5-amino-3-cyclobutyl-1H-pyrazole-1-carboxylate;

ESI (+) MS: m/z 238 (100, MH+); 1H-NMR (400 MHz, DMSO-d6) ppm: 6.27 (s, 1H, H-pyrazole), 2.71 (m, 1H, CH-cyclobutyl), 1.46 (s, 9H, tert-butyl), 2.00-2.34 (2m, 6H, CH2-cyclobutyl).

tertbutyl-5-amino-3-cyclopentyl-1H-pyrazole-1-carboxylate ESI (+) MS: m/z 252 (100, MH+); 1H-NMR (400 MHz, DMSO-d6) ppm: 6.25 (s, 1H, H-pyrazole), 3.10 (m, 1H, CH-cyclopentyl), 1.46 (s, 9H, tert-butyl), 1.52-1.75 (2m, 8H, CH2-cyclopentyl).

(Method B) 1.09 g (10 mmol) o f 3-cyclopropyl-3-oxopropanenitrile and 1.32 g (10 mmol) of tert-butylcarbazate in 25 ml of anhydrous ethanol were stirred at room temperature for 4 hours, then 0.5 ml of triethylamine were added at room temperature. After 3 days under stirring the solution was evaporated to dryness under reduced pressure. The resulting residue was taken up with 100 ml of n-hexane-ethylacetate 70/30 and stirred for 3 hours at 5° C. The solid was filtered through a Buchner finnel and then dried at 40° C. under vacuum yielding 2 g (90%yield) of the title compound.

Following the same method, but employing 3-cyclobutyl- or 3-cyclopentyl-oxopropanenitrile, tertbutyl-5-amino-3-cyclobutyl- or tertbutyl-5-amino-3-cyclopentyl-1H-pyrazole-1-carboxylate can be prepared.

### EXAMPLE 2

### 2-(4-Aminophenyl)propanoic-acid

10 g (0.05 mol) of 2-(4-nitrophenyl)propanoic acid were dissolved in a mixture of 5 ml of water and 100 ml of methanol and 0.65 g of Pd/C 5% were added. The mixture was submitted to hydrogenation at 60 psi for 2 hours at room temperature. After the separation of the catalyst by filtration on celite the methanol was evaporated under vacuum and the title compound was crystallised from water on cooling (7 g; 85% yield).

ESI (+) MS: m/z 166 (100, MH+); 1H-NMR (400 MHz, DMSO-d6) ppm: 7.08-6.71 (2d, 4H, CH-phenyl, J=7.1 Hz), 3.76 (q, 1H, CH-Me, J=7.6 Hz), 1.53 (d, 3H, CH3, 7.6 Hz)

### EXAMPLE 3

### (2S)-2-(4-Aminophenyl)propanoic Acid and (2R)-2-(4-Aminophenyl)propanoic Acid

To 4.34 g of 2-(4-aminophenyl)propanoic acid, 26 ml of water and 3.94 g of L-(+)-tartaric acid were added. The mixture was heated at 80° C. under stirring until complete solution. Heating was then stopped and the solution made to spontaneously cool. After 24 hours 3.8 g of levorotatory tartrate were filtered and dried. The solution containing the dextrorotatory tartrate was concentrated under vacuum at 40° C. to evaporate about 10 ml of water. The solution was then cooled at 30° C. and 1.047 g of sodium hydrate were added. The (+)-(2S)-2-(4-aminophenyl)propanoic acid was collected by filtration and dried to give 1.36 g (α_{D}²⁰=+73.0°; C=0.1% in methanol). Treating the levorotatory tartrate with sodium hydrate the (−)-(2R)-2-(4-aminophenyl)propanoic acid was obtained.

### EXAMPLE 4

### 2-(4-{[(2-Chloroethoxy)carbonyl]amino}phenyl)propanoic Acid

1 g (6 mmol) of 2-(4-aminophenyl)propanoic acid was suspended in 30 ml of dichloromethane and 1.24 ml (12 mmol) of 2-chloroethylchloroformate were added at 0° C. under stirring. After 30 minutes at the same temperature 2.27 g (6 mmol) of sodium phosphate dodecahydrate were added portionwise. After 4 hours the reaction was complete (HPLC-MS). The mixture was made acidic by using hydrochloric acid 0.5N and the product extracted with dichloromethane. The organic layer was dried over sodium sulphate and evaporated under vacuum. 1.4 g (100%yield) of the title compound were obtained by crystallization from diisopropylether.

1H-NMR (400 MHz, DMSO-d6) ppm: 7.47-7.58 (2d, 4H, CH-phenyl, J=9.0 Hz), 3.75 (q, 1H, CH-Me, J=7.6 Hz), 4.31 (m, 2H, CH2Cl), 3.62 (m, 2H, CH2O), 1.53 (d, 3H, CH3, 7.6 Hz)

Analogously, the following intermediates can be prepared starting from the suitable amino derivatives:

(2S)-2-(4-{[(2-chloroethoxy)carbonyl]amino}phenyl)propanoic acid;

(2R)-2-(4-{[(2-chloroethoxy)carbonyl]amino}phenyl)propanoic acid;

4-{[(2-chloroethoxy)carbonyl]amino}phenylacetic acid;

1H-NMR (400 MHz, DMSO-d6) ppm: 7.44-7.60 (2d, 4H, CH-phenyl, J=8.8 Hz), 3.43 (s, 2H, CH2Ph), 4.30 (m, 2H, CH2Cl), 3.63 (m, 2H, CH2O).

### EXAMPLE 5

### 2-[4-(2-oxo-1,3-Oxazolidin-3-yl)phenyl]propanoic Acid

0.5 g (1.85 mmol) of 2-(4-{[(2-chloroethoxy)carbonyl]amino}phenyl)propanoic acid were dissolved in 10 ml of N,N-dimethylformamide and 0.45 g (3.7 mmol) of potassium carbonate were added. After 48 hours the solvent was evaporated under vacuum, the residue redissolved with dichloromethane and washed with hydrochloric acid 0.5 N. The organic layer was dried over sodium sulphate and evaporated. 0.35 g (80% yield) of the title compound crystallized from a mixture ethylacetate-diisopropylether.

ESI (+) MS: m/z 236 (100, MH+); 1H-NMR (400 MHz, DMSO-d6) ppm: 7.08-7.35 (2d, 4H, CH-phenyl, J=8.6 Hz), 3.76 (q, 1H, CH-Me, J=7.6 Hz), 3.80 (m, 2H, CH2N), 4.45 (m, 2H, CH2O), 1.53 (d, 3H, CH3, 7.6 Hz)

Analogously, the following intermediates can be prepared starting from the suitable chloro derivatives:

(2R)-2-[4-(2-oxo-1,3-oxazolidin-3-yl)phenyl]propanoic acid;

(2S)-2-[4-(2-oxo-1,3-oxazolidin-3-yl)phenyl]propanoic acid;

4-(2-oxo-1,3-oxazolidin-3-yl)phenylacetic acid;

ESI (+) MS: m/z 222 (100, MH+); 1H-NMR (400 MHz, DMSO-d6) ppm: 7.10-7.38 (2d, 4H, CH-phenyl, J=8.7 Hz), 3.43 (s, 2H, CH2Ph), 3.79 (m, 2H, CH2N), 4.45 (m, 2H, CH2O).

### EXAMPLE 6

### N-(5-Cyclopropyl-1H-pyrazole-3-yl)-2-[4-(2-oxo-1,3-oxazolidin-3-yl)phenyl]propanamide

3.36 g (14.3 mmol) of 2-[4-(2-oxo-1,3-oxazolidin-3-yl)phenyl]propanoic acid were suspended in 100 ml of dichloromethane. The mixture was cooled to 0° C. and 1.63 ml (18.67 mmol) of oxalyl chloride and 0.5 ml of N,N-dimethylformamide were added.

After 3 hours at room temperature the solvent was evaporated. The resulting acyl chloride, without any further purification, was dissolved in 100 ml of tetrahydrofuran and added dropwise to a solution of 2.87 g (12.87 mmol) of tert-butyl-5-amino-3cyclopropyl-1H-pyrazole-1-carboxylate and 16 ml (85.8 mmol) of N,N-diisopropylethylamine in 80 ml of tetrahydrofuran at 0° C. After 8 hours at room temperature, the solvent was evaporated, the residue redissolved in dichloromethane and washed with a saturated solution of sodium hydrogenocarbonate. The organic layer was dried over sodium sulphate and evaporated under vacuum. The resulting protected amide, without any further purification, was redissolved in 18 ml of dichloromethane and 2 ml of trifluoroacetic acid were added. After 3 hours at room temperature, the solvent was evaporated, the residue taken up with dichloromethane and washed with a sodium hydrogenocarbonate solution. The organic layer was dried over sodium sulfate and evaporated under vacuum. 2 g (41% yield overall) of the title compound were recovered after crystallization with diethylether-ethyl acetate.

ESI (+) MS: m/z 341 (100, MH+); 1H-NMR (400 MHz, DMSO-d6) ppm: 6.88 (m, 4H, CH-phenyl), 6.07 (s, 1H, CH-pyrazole), 3.82 (m, 2H, CH2N), 4.48 (m, 2H, CH2O), 3.85 (q, 1H, CHMe, J=6.8), 1.25 (d, 3H, CH3, J=6.8), 2.27 (m, 1H, CH-cyclopropyl), 1.28-1.35 (2d, 4H, CH2-cyclopropyl).

The following two compounds can be prepared by resolution of their racemic mixture, by chiral preparative HPLC (chiral cel OD; EtOH):

(2R)-N-(5-cyclopropyl-1H-pyrazol-3-yl)-2-[4-(2-oxo-1,3-oxazolidin-3-yl)phenyl]propanamide

(2S)-N-(5-cyclopropyl-1H-pyrazol-3-yl)-2-[4-(2-oxo-1,3-oxazolidin-3-yl)phenyl]propanamide

Analogously the following compound can be prepared starting from the suitable carboxylic acid:

N-(5-cyclopropyl-1H-pyrazol-3-yl)-2-[4-(2-oxo-1,3-oxazolidin-3-yl)phenyl]acetamide ESI (+) MS: m/z 327 (100, MH+);

1H-NMR (400 MHz, DMSO-d6) ppm: 6.92-7.60 (2d, 4H, CH-phenyl, J=8.8 Hz), 6.08 (s, 1H, CH-pyrazole), 3.70 (m, 2H, CH2N), 4.40 (m, 2H, CH2O), 3.68 (s, 2H, CH2Ph), 2.29 (m, 1H, CH-cyclopropyl), 1.26-1.34 (2d, 4H, CH2-cyclopropyl).

Analogously, but using tert-butyl-5-amino-3-cyclobutyl-1H-pyrazole-1-carboxylate the following compounds can be prepared starting from the suitable carboxylic acid:

N-(5-cyclobutyl-1H-pyrazol-3-yl)-2-[4-(2-oxo-1,3-oxazolidin-3-yl)phenyl]propanamide;

(2R)-N-(5-cyclobutyl-1H-pyrazol-3-yl)-2-[4-(2-oxo-1,3-oxazolidin-3-yl)phenyl]propanamide;

(2S)-N-(5-cyclobutyl-1H-pyrazol-3-yl)-2-[4-(2-oxo-1,3-oxazolidin-3-yl)phenyl]propanamide;

N-(5-cyclobutyl-1H-pyrazol-3-yl)-2-[4-(2-oxo-1,3-oxazolidin-3-yl)phenyl]acetamide;

Analogously, but using tert-butyl-5-amino-3-cyclopentyl-1H-pyrazole-1-carboxylate the following compounds can be prepared starting from the suitable carboxilic acid:

N-(5-cyclopentyl-1H-pyrazol-3-yl)-2-[4-(2-oxo-1,3-oxazolidin-3-yl)phenyl]propanamide;

(2R)-N-(5-cyclopentyl-1H-pyrazol-3-yl)-2-[4-(2-oxo-1,3-oxazolidin-3-yl)phenyl]propamide

(2S)-N-(5-cyclopentyl-1H-pyrazol-3-yl)-2-[4-(2-oxo-1,3-oxazolidin-3-yl)phenyl]propamide

N-(5-cyclopentyl-1H-pyrazol-3-yl)-2-[4-(2-oxo-1,3-oxazolidin-3-yl)phenyl]acetamide.

### EXAMPLE 7

### 2-{4-[(tert-Butoxycarbonyl)amino]phenyl}propanoic Acid

12 g (73 mmol) of 2-(4-aminophenyl)propanoic acid were suspended in 140 mL of 1,4-dioxane and 140 ml of water and treated with 7.6 g (72 mmol) of sodium carbonate dissolved in 72 mL of water.

The resulting solution, cooled to 4° C., was treated with 17.2 g (79 mmol) of tert-butoxycarbonyl anhydride and stirred over night allowing the temperature to reach room temperature. The solvent was evaporated, the aqueous phase was washed with ethylacetate, diluted with the same solvent and treated under stirring with 1M potassium hydrogenosulphate. The organic layer was separated and extracted with ethylacetate. The combined organic extracts were washed with brine, dried over sodium sulphate and evaporated. 18.18 g (94% yield) of the title compound were obtained after trituration with hexane.

ESI (+) MS: m/z 266 (100, MH+); 1H-NMR (400 MHz, DMSO-d6) ppm: 7.47-7.52 (m, 4H, CH-phenyl), 3.76 (q, 1H, CHMe, J=7.6 Hz), 1.53 (d, 3H, CH3, J=7.6), 1 51 (s, 9H, tertbutyl).

Analogously, the following compounds can be prepared starting from the suitable carboxylic acid:

(2R)-2-{4-[(tert-butoxycarbonyl)amino]phenyl}propanoic acid;

(2S)-2-{4-[(tert-butoxycarbonyl)amino]phenyl}propanoic acid;

2-{4-[(tert-butoxycarbonyl)amino]phenyl}acetic acid;

ESI (+) MS: m/z 252 (100, MH+); 1H-NMR (400 MHz, DMSO-d6) ppm: 7.45-7.51 (2d, 4H, CH-phenyl, J=8.8 Hz), 3.43 (s, 2H, CH2Ph), 1 51 (s, 9H, tertbutyl).

### EXAMPLE 8

### Tert-Butyl 5-[(2-{4-[(tert-Butoxycarbonyl)amino]phenyl}propanoyl)amino]-3cyclopropyl-1H-pyrazo-e-1-carboxylate

265 mg (1 mmol) of 2-{4-[(tert-butoxycarbonyl)amino]phenyl}propanoic acid were treated at 4° C. under nitrogen with 146 μl (2 mmol) of thionylchloride. The reaction mixture w as stirred for 2.5 hours and the temperature was gradually raised to room temperature. The mixture was taken up with tetrahydrofuran and evaporated thoroughly. The resulting acyl chloride, without any further purification, was dissolved in 3 ml of dry tetrahydrofuran and added dropwise to a solution of 178 mg (0.8 mmol) of tert-butyl-5-amino-3-cyclopropyl-1H-pyrazole-1-carboxylate in 3 ml of tetrahydrofuran. 312 mg (1 mmol, loading 3.2 mmol/g) of polymer supported triethylamine were then added under stirring. After 2.5 hours 80 mg (loading 3.2 mmol/g) of polymer supported trisamine were finally added in order to quench the excess of acyl chloride. After 2 hours, the resins were filtered and washed with methanol and dichloromethane. Evaporation of the filtrate afforded 263 mg of the title compound as a foam (70% yield).

ESI (+) MS: m/z 471 (100, MH+); 1H-NMR (400 MHz, DMSO-d6) ppm: 7.04-7.13 (2d, 4H, CH-phenyl, J=8.5 Hz), 3.62 (s, 1H, CH-pyrazole), 3.73 (q, 1H, CHMe, J=6.9 Hz), 1.28 (d, 3H, CH3, J=6.9 Hz), 1.95 (m, 1H, CH-cyclopropyl), 1.26-1.36 (2d, 4H, CH2-cyclopropyl), 1.51 (s, 9H, tertbutyl-NH), 1.46 (s, 9H, tertbutyl-pyrazole).

Analogously the following compounds can be prepared starting from the suitable carboxylic acid:

(2R)-tert-butyl 5-[(2-{4-[(tert-butoxycarbonyl)amino]phenyl}propanoyl)amino]-3cyclopropyl-1H-pyrazole-1-carboxylate;

(2S)-tert-butyl 5-[(2-{4-[(tert-butoxycarbonyl)amino]phenyl}propanoyl)amino]-3cyclopropyl-1H-pyrazole-1-carboxylate;

tert-butyl 5-[(2-{4-[(tert-butoxycarbonyl)amino]phenyl}acetyl)amino]-3-cyclopropyl-1H-pyrazole-1-carboxylate.

Analogously, but using tert-butyl-5-amino-3-cyclobutyl-1H-pyrazole-1-carboxylate the following compounds can be prepared starting from the suitable carboxylic acid:

tert-butyl 5-[(2-{4-[(tert-butoxycarbonyl)amino]phenyl}propanoyl)amino]-3cyclobutyl-1H-pyrazole-1-carboxylate;

(2R)-tert-butyl 5-[(2-{4-[(tert-butoxycarbonyl)amino]phenyl}propanoyl)amino]-3cyclobutyl-1H-pyrazole-1-carboxylate;

(2S)-tert-butyl 5-[(2-{4-[(tert-butoxycarbonyl)amino]phenyl}propanoyl)amino]-3cyclobutyl-1H-pyrazole-1-carboxylate;

tert-butyl 5-[(2-{4-[(tert-butoxycarbonyl)amino]phenyl}acetyl)amino]-3-cyclobutyl-1H-pyrazole-1-carboxylate.

Analogously, but using tert-butyl-5-amino-3-cyclopentyl-1H-pyrazole-1-carboxylate the following compounds can be prepared starting from the suitable carboxylic acid:

tert-butyl 5-[(2-{4-[(tert-butoxycarbonyl)amino]phenyl}propanoyl)amino]-3-cyclopentyl-1H-pyrazole-1-carboxylate;

(2R)-tert-butyl 5-[(2-{4-[(tert-butoxycarbonyl)amino]phenyl}propanoyl)amino]-3-cyclopentyl-1H-pyrazole-1-carboxylate;

(2S)-tert-butyl 5-[(2-{4-[(tert-butoxycarbonyl)amino]phenyl}propanoyl)amino]-3-cyclopentyl-1H-pyrazole-1-carboxylate;

tert-butyl 5-[(2-{4-[(tert-butoxycarbonyl)amino]phenyl}acetyl)amino]-3-cyclopentyl-1H-pyrazole-1-carboxylate.

### EXAMPLE 9

### 2-(4-Aminophenyl)-N-(5-cyclopropyl-1H-pyrazol-3-yl)propanamide

11.8 g (25.26 mmol) of tert-butyl 5-[(2-{4-[(tert-butoxycarbonyl)amino]phenyl}propanoyl)amino]-3-cyclopropyl-1H-pyrazole-1-carboxylate were dissolved in 200 ml of dichloromethane and 30 ml of trifluoroacetic acid were added. After 2.5 hours at room temperature the solvent was evaporated and the residue taken up with dichloromethane and washed with a saturated solution of sodium hydrogenocarbonate. The organic layer was then dried over sodium sulphate and evaporated to dryness, affording 4.7 g (70% yield) of the title compound.

ESI (+) MS: m/z 271 (100,MH+); 1H-NMR (400 MHz, DMSO-d6) ppm: 6.62 (s, 4H, CH-phenyl), 6.07 (s, 1H, CH-pyrazole), 3.82 (q, 1H, CHMe, J=6.8 Hz), 1.25 (d, 3H, CH3, J=6.8 Hz), 2.27 (m, 1H, CH-cyclopropyl), 1.28-1.35 (2d, 4H, CH2-cyclopropyl).

Analogously, the following compounds can be prepared starting from the suitable carboxylic acid:

(2R)-2-(4-aminophenyl)-N-(5-cyclopropyl-1H-pyrazol-3-yl)propanamide;

(2S)-2-(4-aminophenyl)-N-(5-cyclopropyl-1H-pyrazol-3-yl)propanamide;

2-(4-aminophenyl)-N-(5-cyclopropyl-1H-pyrazol-3-yl)acetamide;

ESI (+) MS: m/z 258 (100,MH+); 1H-NMR (400 MHz, DMSO-d6) ppm: 6.61-7.34 (2d, 4H, CH-phenyl, J=8.8 Hz), 6.08 (s, 1H, CH-pyrazole), 3.67 (s, 2H, CH2Ph), 2.27 (m, 1H, CH-cyclopropyl), 1.28-1.35 (2d, 4H, CH2-cyclopropyl).

Analogously, but using tert-butyl-5-amino-3-cyclobutyl-1H-pyrazole-1-carboxylate the following compounds can be prepared starting from the suitable carboxylic acid:

2-(4-aminophenyl)-N-(5-cyclobutyl-1H-pyrazol-3-yl)propanamide;

(2R)-2-(4-aminophenyl)-N-(5-cyclobutyl-1H-pyrazol-3-yl)propanamide;

(2S)-2-(4-aminophenyl)-N-(5-cyclobutyl-1H-pyrazol-3-yl)propanamide;

2-(4-aminophenyl)-N-(5-cyclobutyl-1H-pyrazol-3-yl)acetamide;

Analogously, but using tert-butyl-5-amino-3-cyclopentyl-1H-pyrazole-1-carboxylate the following compounds can be prepared starting from the suitable carboxylic acid:

2-(4-aminophenyl)-N-(5-cyclopentyl-1H-pyrazol-3-yl)propanamide;

(2R)-2-(4-aminophenyl)-N-(5-cyclopentyl-1H-pyrazol-3-yl)propanamide;

(2S)-2-(4-aminophenyl)-N-(5-cyclopentyl-1H-pyrazol-3-yl)propanamide;

2-(4-aminophenyl)-N-(5-cyclopentyl-1H-pyrazol-3-yl)acetamide.

### EXAMPLE 10

### 2-Chloroethyl 5-{[2-(4-{[(2-chloroethoxy)carbonyl]amino}phenyl)propanoyl]amino}-3-cyclopropyl-1H-pyrazole-1-carboxylate

154 mg (0.57 mmol) of 2-(4-aminophenyl)-N-(5-cyclopropyl-1H-pyrazol-3-yl)propanamide were dissolved in 1.9 ml of dry pyridine at 4° C. under argon atmosphere and 0.12 ml (1.148 mmol) of 2-chloroethylchloroformate were added dropwise. After 2.5 hours the reaction mixture was poured into ice and the precipitate solid collected by filtration, affording 225 mg (82% yield) of the title compound.

1H-NMR (400 MHz, DMSO-d6) ppm: 7.37-7.41 (m, 4H, CH-phenyl), 3.32 (s, 1H, CH-pyrazole), 3.85 (q, 1H, CHMe, J=6.8 Hz), 1.24 (d, 3H, CH3, J=6.8 Hz), 1.93 (m, 1H, CH-cyclopropyl), 1.27-1.35 (2d, 4H, CH2-cyclopropyl), 3.63-3.85 (m, 4H, CH2O), 4.30 (m, 4H, CH2Cl).

Analogously, but employing the suitable amide derivatives, the following compounds can be prepared:

2-chloroethyl 5-{[(2R)-2-(4-{[(2-chloroethoxy)carbonyl]amino}phenyl)propanoyl]amino}-3-cyclopropyl-1H-pyrazole-1-carboxylate;

2-chloroethyl 5-{[(2S)-2-(4-{[(2-chloroethoxy)carbonyl]amino}phenyl)propanoyl]amino}-3-cyclopropyl-1H-pyrazole-1-carboxylate;

2-chloroethyl 5-{[2-(4-{[(2-chloroethoxy)carbonyl]amino}phenyl)acetyl]amino}-3cyclopropyl-1H-pyrazole-1-carboxylate;

2-chloroethyl 5-{[(2R)-2-(4-{[(2-chloroethoxy)carbonyl]amino}phenyl)propanoyl]amino}-3-cyclobutyl-1H-pyrazole-1-carboxylate;

2-chloroethyl 5-{[(2S)-2-(4-{[(2-chloroethoxy)carbonyl]amino}phenyl)propanoyl]amino}-3-cyclobutyl-1H-pyrazole-1-carboxylate;

2-chloroethyl 5-{[2-(4-{[(2-chloroethoxy)carbonyl]amino}phenyl)propanoyl]amino}3-cyclobutyl-1H-pyrozole-1-carboxylate;

2-chloroethyl 5-{[2-(4-{[(2-chloroethoxy)carbonyl]amino}phenyl)acetyl]amino}-3-cyclbutyl-1H-pyrazole-1-carboxylate;

2-chloroethyl 5-{[(2R)-2-(4-{[(2-chloroethoxy)carbonyl]amino}phenyl)propanoyl]amino}-3-cyclopentyl-1H-pyrazole-1-carboxylate;

2-chloroethyl 5-{[(2S)-2-(4-{[(2-chloroethoxy)carbonyl]amino}phenyl)propanoyl]amino}-3-cyclopentyl-1H-pyrazole-1-carboxylate;

2-chloroethyl 5-{[2-(4-{[(2-chloroethoxy)carbonyl]amino}phenyl)propanoyl]amino}-1H-pyrazole-1-carboxylate;

2-chloroethyl 5-{[2-(4-{[(2-chloroethoxy)carbonyl]amino}phenyl)acetyl]amino}-3cyclopentyl-1H-pyrazole-1-carboxylate.

### EXAMPLE 11

### N-(3-Cyclopropyl-1H-pyrazol-5-yl)-2-[4-(2-oxo-1,3-oxazolidin-3-yl)phenyl]propanamide

431 mg (0.89 mmol) of 2-chloroethyl 5-{[2-(4-{[(2-chloroethoxy)carbonyl]amino}phgenyl)propanoyl]amino}-3-cyclopropyl-1H-pyrazole-1-carboxylate were dissolved in 3 ml of N,N-dimethylformamide and 124 m g (0.89 mmol) of potassium carbonate were added and the mixture stirred at room temperature for 19 hours. The reaction mixture was poured into 30 ml of methanol and stirred for 40 minutes, evaporated under vacuum, diluted with 40 ml of m ethanol and washed with a saturated aqueous ammonium chloride solution. The organic layer was further extracted with dichloromethane, dried over sodium sulfate and evaporated to dryness. The crude was purified by chromatography on a silica gel column to afford 233 mg (77 % yield) of the title compound.

Analogously, but employing the suitable bisacylated amide derivatives, the following compounds can be prepared:

(2R)-N-(3-cyclopropyl-1H-pyrazol-5-yl)-2-[4-(2-oxo-1,3-oxazolidin-3-yl)phenyl]propanamide

(2S)-N-(3-cyclopropyl-1H-pyrazol-5-yl)-2-[4-(2-oxo-1,3-oxazolidin-3-yl)phenyl]propanamide;

N-(3-cyclopropyl-1H-pyrazol-5-yl)-2-[4-(2-oxo-1,3-oxazolidin-3-yl)phenyl]acetamide;

(2R)-N-(3-cyclobutyl-1H-pyrazol-5-yl)-2-[4-(2-oxo-1,3-oxazolidin-3-yl)phenyl]propanamide;

(2S)-N-(3-cyclobutyl-1H-pyrazol-5-yl)-2-[4-(2-oxo-1,3-oxazolidin-3-yl)phenyl]propanamide;

N-(3-cyclobutyl-1H-pyrazol-5-yl)-2-[4-(2-oxo-1,3-oxazolidin-3-yl)phenyl]propanamide

N-(3-cyclobutyl-1H-pyrazol-5-yl)-2-[4-(2-oxo-1,3-oxazolidin-3-yl)phenyl]acetamide;

N-(3-cyclopentyl-1H-pyrazol-5-yl)-2-[4-(2-oxo-1,3-oxazolidin-3-yl)phenyl]propanamide

(2R)-N-(3-cyclopentyl-1H-pyrazol-5-yl)-2-[4-(2-oxo-1,3-oxazolidin-3-yl)phenyl]propanamide;

(2S)-N-(3-cyclopentyl-1H-pyrazol-5-yl)-2-[4-(2-oxo-1,3-oxazolidin-3-yl)phenyl]propanamide;

N-(3-cyclopentyl-1H-pyrazol-5-yl)-2-[4-(2-oxo-1,3-oxazolidin-3-yl)phenyl]acetamide.

### EXAMPLE 12

### N-(3-Cyclopropyl-1H-pyrazole-5-yl)-2-14-(2-oxo-1-pyrrolidinyl)phenyl]propanamide

1.29 g (5.56 mmol) of 2-[4-(2-oxo-1-pyrrolidinyl)phenyl]propanoic acid were dissolved in 20 ml of dichloromethane and 0.95 ml (5.56 mmol) of N,N-diisopropylethylamine and 1.06 g (5.56 mmol) of 1-(3-dimethylaminopropyl)-3-ethylcarbodiimide were added under stirring at 0° C. After 20 minutes at the same temperature 0.62 g (2.78 mmol) of tertbutyl-5-amino-3-cyclopropyl-1H-pyrazole-1-carboxylate dissolved in 10 ml of dichloromethane were added dropwise. After 12 hours at room temperature, the solution was washed with a sodium hydrogenocarbonate saturated solution, dried over anhydrous sodium sulphate and concentrated in vacuo to give 0.85 g (70% yield) of tertbutyl-3-cyclopropyl-5-({2-[4(2-oxo-1-pyrrolidinyl)phenyl]propanoyl} amino)-1H-pyrazole-1-carboxylate. Without any further purification this intermediate was redissolved in 40 ml of ethanol and 2 ml of 10% sulphuric acid were added. After 12 hours at room temperature under stirring the solution was neutralized with sodium hydrogenocarbonate, the ethanol evaporated and the resulting precipitate collected, washed with water and dessiccated in vacuo to give 590 mg of the title compound (90% yield).

By working in an analogous way and by using the suitable carboxylic acid, the following compounds can be prepared:

N-(5-cyclopropyl-1H-pyrazol-3-yl)-2-fluoro-2-[4-(2-oxo-1-pyrrolidinyl)phenyl]acetamide;

2-chloro-N-(5-cyclopropyl-1H-pyrazol-3-yl)-2-[4-(2-oxo-1-pyrrolidinyl)phenyl]acetamide;

N-(5-cyclopropyl-1H-pyrazol-3-yl)-2,2-difluoro-2-[4-(2-oxo-1-pyrrolidinyl)phenyl]acetamide;

N-(5-cyclopropyl-1H-pyrazol-3-yl)-3,3,3-trifluoro-2-[4-(2-oxo-1-pyrrolidinyl)phenyl]acetamide;

3-amino-N-(5-cyclopropyl-1H-pyrazol-3-yl)-2-[4-(2-oxo-1-pyrrolidinyl)phenyl]propanamide;

N-(5-cyclopropyl-1H-pyrazol-3-yl)-2-[4-(3-methyl-2-oxo-1-pyrrolidinyl)phenyl]propanamide

N-(5-cyclopropyl-1H-pyrazol-3-yl)-2-[4-(2-methyl-5-oxo-1-pyrrolidinyl)phenyl]propanamide

N-(5-cyclopropyl-1H-pyrazol-3-yl)-2-[4-(2-ethyl-5-oxo-1-pyrrolidinyl)phenyl]propanamide;

N-(5-cyclopropyl-1H-pyrazol-3-yl)-2-[4-(2-oxo-5-phenyl-1-pyrrolidinyl)phenyl]propanamide;

N-(5-cyclopropyl-1H-pyrazol-3-yl)-2-[4-(2-oxo-3,3a,6,6a-tetrahydrocyclopenta[b]pyrrol-1(2H)-yl)phenyl]propanamide;

N-(5-cyclopropyl-1H-pyrazol-3-yl)-2-{4-[2-(hydroxymethyl)-5-oxo-1-pyrrolidinyl]phenyl}propanamide;

N-(5-cyclopropyl-1H-pyrazol-3-yl)-2-[4-(3-hydroxy-2-oxo-1-pyrrolidinyl)phenyl]propanamide;

N-(5-cyclopropyl-1H-pyrazol-3-yl)-2-[4-(4-hydroxy-2-oxo-1-pyrrolidinyl)phenyl]propanamide;

N-(5-cyclopropyl-1H-pyrazol-3-yl)-2-[4-(3-hydroxy-4,4-dimethyl-2-oxo-1-pyrrolidinyl)phenyl]propanamide;

1-(4-{2-[(5-cyclopropyl-1H-pyrazol-3-yl)amino]-1-methyl-2-oxoethyl}phenyl)-2-oxo-3-pyrrolidinecarboxamide;

1-(4-{2-[(5-cyclopropyl-1H-pyrazol-3-yl)amino]-1-methyl-2-oxoethyl}phenyl)-5-oxo-3-pyrrolidinecarboxamide;

N-(5-cyclopropyl-1H-pyrazol-3-yl)-2-[4-(2,5-dioxo-2,5-dihydro-1H-pyrrol-1-yl)phenyl]propanamide;

N-(5-cyclopropyl-1H-pyrazol-3-yl)-2-[4-(2,5-dioxo-1-pyrrolidinyl)phenyl]propanamide;

N-(5-cyclopropyl-1H-pyrazol-3-yl)-2-[4-(6-methoxy-1-oxo-1,3-dihydro-2H-isoindol-2-yl)phenyl]propanamide;

N-(5-cyclopropyl-1H-pyrazol-3-yl)-2-[4-(1-oxo-1H-benzo[de]isoquinolin-2(3H)-yl)phenyl]propanamide;

N-(5-cyclopropyl-1H-pyrazol-3-yl)-2-[4-(1H-pyrrol-1-yl)phenyl]propanamide;

N-(5-cyclopropyl-1H-pyrazol-3-yl)-2-[4-(2,4-dioxo-1-imidazolidinyl)phenyl]propanamide;

N-(5-cyclopropyl-1H-pyrazol-3-yl)-2-[4-(2-oxo-1-imidazolidinyl)phenyl]propanamide;

N-(5-cyclopropyl-1H-pyrazol-3-yl)-2-[4-(2-oxo-2,3-dihydro-1H-imidazol-1-yl)phenyl]propanamide;

N-(5-cyclopropyl-1H-pyrazol-3-yl)-2-[4-(5-oxo-1,5-dihydro-4H-1,2,4-triazol-4-yl)phenyl]propanamide;

1-(4-{2-[(5-cyclopropyl-1H-pyrazol-3-yl)amino]-1-methyl-2-oxoethyl}phenyl)-5-hydroxy-1H-pyrazole-3-carboxamide;

N-(5-cyclopropyl-1H-pyrazol-3-yl)-2-[4-(3-oxo-1-pyrazolidinyl)phenyl]propanamide;

N-(5-cyclopropyl-1H-pyrazol-3-yl)-2-[4-(3,5-dioxo-1-pyrazolidinyl)phenyl]propanamide;

N-(5-cyclopropyl-1H-pyrazol-3-yl)-2-[4-(2,4-dioxo-3,4-dihydro-1(2H)-pyrimidinyl)phenyl]propanamide;

N-(5-cyclopropyl-1H-pyrazol-3-yl)-2-[4-(3,5-dioxo-4morpholinyl)phenyl]propanamide;

N-(5-cyclopropyl-1H-pyrazol-3-yl)-2-[4-(2-oxo-1-piperidinyl)phenyl]propanamide;

N-(5-cyclopropyl-1H-pyrazol-3-yl)-2-[4-(1-piperazinyl)phenyl]propanamide;

N-(5-cyclopropyl-1H-pyrazol-3-yl)-2-[4-(4-morpholinyl)phenyl]propanamide;

N-(5-cyclopropyl-1H-pyrazol-3-yl)-2-[4-(1-pyrrolidinyl)phenyl]propanamide;

N-(5-cyclopropyl-1H-pyrazol-3-yl)-2-[4-(1-piperidinyl)phenyl]propanamide.

(2S)-N-(5-cyclopropyl-1H-pyrazol-3-yl)-2-[4-(2-oxo-1-pyrrolidinyl)phenyl]propanamide;

(2R)-N-(5-cyclopropyl-1H-pyrazol-3-yl)-2-[4-(2-oxo-1-pyrrolidinyl)phenyl]propanamide;

(2S)-N-(5-cyclobutyl-1H-pyrazol-3-yl)-2-[4-(2-oxo-1-pyrrolidinyl)phenyl]propanamide;

(2R)-N-(5-cyclobutyl-1H-pyrazol-3-yl)-2-[4-(2-oxo-1-pyrrolidinyl)phenyl]propanamide;

N-(5-cyclopropyl-1H-pyrazol-3-yl)-2-fluoro-2-[4-(2-oxo-1-pyrrolidinyl)phenyl]acetamide;

2-chloro-N-(5-cyclopropyl-1H-pyrazol-3-yl)-2-[4-(2-oxo-1-pyrrolidinyl)phenyl]acetamide;

N-(5-cyclopropyl-1H-pyrazol-3-yl)-2,2-difluoro-2-[4-(2-oxo-1-pyrrolidinyl)phenyl]acetamide;

N-(5-cyclopropyl-1H-pyrazol-3-yl)-2-[4-(3-methyl-2-oxo-1-pyrrolidinyl)phenyl]acetamide;

N-(5-cyclopropyl-1H-pyrazol-3-yl)-2-[4-(3-methyl-2-oxo 1-pyrrolidinyl)phenyl]acetamide;

N-(5-cyclopropyl-1H-pyrazol-3-yl)-2-[4-(2-methyl-5-oxo-1-pyrrolidinyl)phenyl]acetamide;

N-(5-cyclopropyl-1H-pyrazol-3-yl)-2-[4-(2-ethyl-5-oxo-1-pyrrolidinyl)phenyl]acetamide;

N-(5-cyclopropyl-1H-pyrazol-3-yl)-2-[4-(2-oxo-5-phenyl-1-pyrrolidinyl)phenyl]acetamide;

N-(5-cyclopropyl-1H-pyrazol-3-yl)-2-[4-(2-oxo-3,3a,6,6a-tetrahydrocyclopenta[b]pyrrol-1(2H)-yl)phenyl]acetamide;

N-(5-cyclopropyl-1H-pyrazol-3-yl)-2-{4-[2-(hydroxymethyl)-5-oxo-1-pyrrolidinyl]phenyl}acetamide;

N-(5-cyclopropyl-1H-pyrazol-3-yl)-2-[4-(3-hydroxy-2-oxo-1-pyrrolidinyl)phenyl]acetamide;

N-(5-cyclopropyl-1H-pyrazol-3-yl)-2-[4-(4-hydroxy-2-oxo-1-pyrrolidinyl)phenyl]acetamide;

N-(5-cyclopropyl-1H-pyrazol-3-yl)-2-[4-(3-hydroxy-4,4-dimethyl-2-oxo-1-pyrrolidinyl)phenyl]acetamide;

1-(4-{2-[(5-cyclopropyl-1H-pyrazol-3-yl)amino]-2-oxoethyl}phenyl)-2-oxo-3-pyrrolidinecarboxamide;

1-(4-{2-[(5-cyclopropyl-1H-pyrazol-3-yl)amino]-2-oxoethyl}phenyl)-5-oxo-3-pyrrolidinecarboxamide;

1-(4-{2-[(5-cyclopropyl-1H-pyrazol-3-yl)amino]-2-oxoethyl}phenyl)-5-oxo-2-pyrrolidinecarboxamide;

N-(5-cyclopropyl-1H-pyrazol-3-yl)-2-[4-(2,5-dioxo-1-pyrrolidinyl)phenyl]acetamide;

N-(5-cyclopropyl-1H-pyrazol-3-yl)-2-[4-(2,4-dioxotetrahydro-1(2H)-pyrimidinyl)phenyl]acetamide;

N-(5-cyclopropyl-1H-pyrazol-3-yl)-2-[4-(3,5-dioxo-1,2,4-triazolidin-4-yl)phenyl]acetatamide;

N-(5-cyclopropyl-1H-pyrazol-3-yl)-2-[4-(2,5-dioxo-1-imidazolidinyl)phenyl]acetamide;

N-(5-cyclopropyl-1H-pyrazol-3-yl)-2-[4-(2,4-dioxo-1-imidazolidinyl)phenyl]acetamide;

N-(5-cyclopropyl-1H-pyrazol-3-yl)-2-[4-(2-oxo-1-imidazolidinyl)phenyl]acetamide;

N-(5-cyclopropyl-1H-pyrazol-3-yl)-2-[4-(2-oxo-2,3-dihydro-1H-imidazol-1-yl)phenyl]acetamide;

N-(5-cyclopropyl-1H-pyrazol-3-yl)-2-[4-(5-oxo-1,5-dihydro-4H-1,2,4-triazol-4-yl)phenyl]acetamide;

1-(4-{2-[(5-cyclopropyl-1H-pyrazol-3-yl)amino]-2-oxoethyl}phenyl)-5-hydroxy-1H-pyrazole-3-carboxamide;

N-(5-cyclopropyl-1H-pyrazol-3-yl)-2-[4-(3-oxo-1-pyrazolidinyl)phenyl]acetamide;

N-(5-cyclopropyl-1H-pyrazol-3-yl)-2-[4-(3,5-dioxo-1-pyrazolidinyl)phenyl]acetamide;

N-(5-cyclopropyl-1H-pyrazol-3-yl)-2-[4-(2,4-dioxo-3,4-dihydro-1(2H)-pyrimidinyl)phenyl]acetamide;

N-(5-cyclopropyl-1H-pyrazol-3-yl)-2-[4-(3,5-dioxo-4-morpholinyl)phenyl]acetamide;

N-(5-cyclopropyl-1H-pyrazol-3-yl)-2-[4-(2-oxo-1-piperidinyl)phenyl]acetamide;

N-(5-cyclopropyl-1H-pyrazol-3-yl)-2-[4-(1-piperazinyl)phenyl]acetamide;

N-(5-cyclopropyl-1H-pyrazol-3-yl)-2-[4-(4-morpholinyl)phenyl]acetamide;

N-(5-cyclopropyl-1H-pyrazol-3-yl)-2-[4-(1-piperidinyl)phenyl]acetamide;

### EXAMPLE 13

### 2-[4-(2-oxo-1-Pyrrolidinyl)phenyl]propanoic Acid

20.8 ml (0.27 mol) of γ-butirolactone and 11 ml of 37% hydrochloric acid were added to 30 g (0.18 mol) of 2-(4-aminophenyl)propanoic acid. The mixture was heated at 180° C. overnight. After cooling to about 50° C. 200 ml of 2N hydrochloric acid were added dropwise under vigorous stirring and the product was collected by filtration and dessiccated under vacuum at 50° C., giving 13 g (32% yield) of the title compound.

By working in an analogous way and by employing the suitable lactone derivative, the following compounds can be prepared:

2-[4-(2-oxo-1-piperidinyl)phenyl]propanoic acid;

2-[4-(3-methyl-2-oxo-1-pyrrolidinyl)phenyl]propanoic acid;

2-[4-(2-methyl-5-oxo-1-pyrrolidinyl)phenyl]propanoic acid;

2-[4-(2-ethyl-5-oxo-1-pyrrolidinyl)phenyl]propanoic acid;

2-[4-(2-oxo-3,3a,6,6a-tetrahydrocyclopenta[b]pyrrol-1(2H)-yl)phenyl]propanoic acid;

2-[4-(2-oxo-5-phenyl-1-pyrrolidinyl)phenyl]propanoic acid;

2-{4-[2-(hydroxymethyl)-5-oxo-1-pyrrolidinyl]phenyl}propanoic acid;

2-[4-(3-hydroxy-2-oxo-1-pyrrolidinyl)phenyl]propanoic acid;

2-[4-(4-hydroxy-2-oxo-1-pyrrolidinyl)phenyl]propanoic acid;

2-[4-(3-hydroxy-4,4-dimethyl-2-oxo-1-pyrrolidinyl)phenyl]propanoic acid;

2-{4-[2-(aminocarbonyl)-5-oxo-1-pyrrolidinyl]phenyl}propanoic acid.

By working in an analogous way and by employing 2-(4-aminophenyl)acetic acid and the suitable lactone derivative, the following compounds can be prepared:

[4-(2-oxo-1-piperidinyl)phenyl]acetic acid;

[4-(3-methyl-2-oxo-1-pyrrolidinyl)phenyl]acetic acid;

[4-(2-methyl-5-oxo-1-pyrrolidinyl)phenyl]acetic acid;

[4-(2-ethyl-5-oxo-1-pyrrolidinyl)phenyl]acetic acid;

[4-(2-oxo-3,3a,6,6a-tetrahydrocyclopenta[b]pyrrol-1(2H)-yl)phenyl]acetic acid;

[4-(2-oxo-5-phenyl-1-pyrrolidinyl)phenyl]acetic acid;

{4-[2-(hydroxymethyl)-5-oxo-1-pyrrolidinyl]phenyl}acetic acid;

[4-(3-hydroxy-2-oxo-1-pyrrolidinyl)phenyl]acetic acid;

[4-(3-hydroxy-4,4-dimethyl-2-oxo-1-pyrrolidinyl)phenyl]acetic acid;

[4-(4-hydroxy-2-oxo-1-pyrrolidinyl)phenyl]acetic acid;

{4-[2-(aminocarbonyl)-5-oxo-1-pyrrolidinyl]phenyl}acetic acid

### EXAMPLE 14

### Methyl 2-(4-Aminophenyl)propanoate

10 g (0.06 mol) of 2-(4-aminophenyl)propanoic acid were dissolved in 100 ml of methanol and 6.5 ml of 96% sulphuric acid were added dropwise at 0° C. After 6 hours the methanol was evaporated and the residue poured into icy water. The solution was then basified with 30% ammonium hydrate and extracted with dichloromethane, giving, after treatment with anhydrous sodium sulfate and concentration under vacuum, 9.6 g (90%yield) of the title compound.

By working in an analogous way and by employing 2-(4-aminophenyl)acetic acid, methyl 2-(4-aminophenyl)acetate can be prepared.

### EXAMPLE 15

### Methyl 2-[4-(1-Pyrrolidinyl)phenyl]propanoate

17.9 g (0.1 mol) of methyl 2-(4-aminophenyl)propanoate were dissolved in 450 ml of N,N.dimethylformamide and 15.3 (0.13 mol) of 1,4-dibromobutane and 69.6 ml (0.4 mol) of N,N-diisopropylethylamine were added. The mixture was heated under stirring at 90° C. for 5 hours. The solvent was then evaporated under vacuum, the residue redissolved in dichloromethane and the resulting solution washed with water, dried over anhydrous sodium sulphate and concentrated. The crude was finally purified on a Florisil (200-300 mesh) column by using n-hexane as eluant, leading 14 g (60% yield) of the title compound.

### EXAMPLE 16

### 2-[4-(1-Pyrrolidinyl)phenyl]propanoic Acid

2 g (8.5 mmol) of methyl 2-[4-(1-pyrrolidinyl)phenyl]propanoate were dissolved in 70 ml of glacial acetic acid and 28 ml of 2N hydrochloric acid were added. The mixture was heated at 120° C. for 4 hours and then evaporated in vacuo. The residue was redissolved in water and 8 ml of 2N sodium hydrate were added. The precipitate title compound (1.8 g; 90% yield) was collected by filtration, washed with water and dessiccated.

### EXAMPLE 17

### N-(5-Cyclopropyl-1H-pyrazol-3-yl)-2-[4-(1-pyrrolidinyl)phenyl]propanamide

1 g (4.5 mmol) of 2-[4-(1-pyrrolidinyl)phenyl]propanoic acid was dissolved in 30 ml of dichloromethane and 1 ml of N,N-dimethylformamide and 0.51 ml of oxalyl chloride in 15 ml of dichloromethane were added dropwise. After 30 minutes at room temperature the solution was evaporated in vacuo to give an orange solid, that was redissolved in 30 ml of dichloromethane and added dropwise to a solution of 0.92 g (4.12 mmol) of tertbutyl-3-amino-5-cyclopropyl-1H-pyrazole-1-carboxylate and 0.65 (4.5 mmol) of triethylamine in 15 of dichloromethane at 0° C. under stirring. The mixture was maintained at room temperature overnight, then washed with 5% citric acid solution, a saturated aqueous sodium hydrogenocarbonate solution and finally brine. The organic layer was dried over anhydrous sodium sulphate and evaporated to give 1.76 g (89% of tertbutyl-5-cyclopropyl-3-({2-[4-(1-pyrrilidinyl)phenyl]propanoyl}amino)-1H-pyrazole-1-carboxylate. This intermediate was dissolved in 66 ml of dichloromethane and 6.6 ml of trifluoroacetic acid were added. After 2 hours at room temperature the solvent was evaporated and the residue redissolved in dichloromethane and washed with aqueous sodium hydrogenocarbonate. The organic layer was then dried over sodium sulphate and concentrated. The crude was then purified by chromatography on a silica gel column by using dichloromethane-ethylacetate 7/3 as eluant, giving 915 mg of the title compound (68% yield).

By working in an analogous way and by employing the suitable carboxylic acid, the following compounds can be prepared:

N-(5-cyclopropyl-1H-pyrazol-3-yl)-2-[4-(1,3-dioxo-1,3-dihydro-2H-isoindol-2-yl)phenyl]propanamide;

N-(5-cyclopropyl-1H-pyrazol-3-yl)-2-[4-(6-methoxy-1-oxo-1,3-dihydro-2H-isoindol-2-yl)phenyl]propanamide;

N-(5-cyclopropyl-1H-pyrazol-3-yl)-2-[4-(1,1-dioxido-3-oxo-1,2-benzisothiazol-2(3H)-yl)phenyl]propanamide;

N-(5-cyclopropyl-1H-pyrazol-3-yl)-2-[4-(2,5-dioxo-2,5-dihydro-1H-pyrrol-1-yl)phenyl]propanamide;

N-(5-cyclopropyl-1H-pyrazol-3-yl)-2-[4-(1-oxo-1,3-dihydro-2H-benzo[f]isoindol-2-yl)phenyl]propanamide;

N-(5-cyclopropyl-1H-pyrazol-3-yl)-2-[4-(1-oxo-1H-benzo[de]isoquinolin-2(3H)-yl)phenyl]propanamide;

N-(5-cyclopropyl-1H-pyrazol-3-yl)-2-[4-(1H-pyrrol-1-yl)phenyl]propanamide;

N-(5-cyclopropyl-1H-pyrazol-3-yl)-2-[4-(3H-[ 1,2,3]triazolo[4,5-d]pyrimidin-3-yl)phenyl]propanamide;

2-[2-chloro-4-(1-oxo-1,3-dihydro-2H-isoindol-2-yl)phenyl]-N-(5-cyclopropyl-1H-pyrazol-3-yl)propanmide;

N-(5-cyclopropyl-1H-pyrazol-3-yl)-2-[4-(1-hydroxy-3-oxo-1,3-dihydro-2H-isoindol-2-yl)phenyl]acetamide;

N-(5-cyclopropyl-1H-pyrazol-3-yl)-2-[4-(1,3-dioxo-1,3-dihydro-2H-isoindol-2-yl)phenyl]acetamide;

N-(5-cyclopropyl-1H-pyrazol-3-yl)-2-[4-(1-oxooctahydro-2H-isoindol-2-yl)phenyl]acetamide;

N-(5-cyclopropyl-1H-pyrazol-3-yl)-2-[4-(2,5-dioxo-2,5-dihydro-1H-pyrrol-1-yl)phenyl]acetamide;

N-(5-cyclopropyl-1H-pyrazol-3-yl)-2-[4-(6-methoxy-1-oxo-1,3-dihydro-2H-isoindol-2-yl)phenyl]propanemide;

N-(5-cyclopropyl-1H-pyrazol-3-yl)-2-[4-(1,1-dioxido-3-oxo-1,2-benzisothiazol-2(3H)-yl)phenyl]acetamide;

N-(5-cyclopropyl-1H-pyrazol-3-yl)-2-[4-(1H-pyrrol-1-yl)phenyl]acetamide;

N-(5-cyclopropyl-1H-pyrazol-3-yl)-2-[4-(7-hydroxy-3H-[ 1,2,3]triazolo[4,5-d]pyrimidin-3-yl)phenyl]acetamide;

2-[2-chloro-4-(1-oxo-1,3-dihydro-2H-isoindol-2-yl)phenyl]-N-(5-cyclopropyl-1H-pyrazol-3-yl)acetamide;

The following intermediates, employed to give the corresponding compounds of formula (I), were prepared according to known methods:

2-[4-(1,3-dioxo-1,3-dihydro-2H-benzo[f]isoindol-2-yl)phenyl]propanoic acid;

2-[4-(1-oxo-1H-benzo[de]isoquinolin-2(3H)-yl)phenyl]propanoic acid;

2-[4-(1H-pyrrol-1-yl)phenyl]propanoic acid;

2-[4-(7-hydroxy-3H-[1,2,3]triazolo[4,5-d]pyrimidin-3-yl)phenyl]propanoic acid;

2-[2-chloro-4-(1-oxo-1,3-dihydro-2H-isoindol-2-yl)phenyl]propanoic acid;

2-[4-(1-oxooctahydro-2H-isoindol-2-yl)phenyl]propanoic acid;

2-[4-(1-hydroxy-3-oxo-1,3-dihydro-2H-isoindol-2-yl)phenyl]propanoic acid;

2-[4-(2,5-dioxo-2,5-dihydro-1H-pyrrol-1-yl)phenyl]propanoic acid;

2-[4-(2,5-dioxo-1-pyrrolidinyl)phenyl]propanoic acid;

2-[4-(6-methoxy-1-oxo-1,3-dihydro-2H-isoindol-2-yl)phenyl]propanoic acid;

2-[4-(1,1-dioxido-3-oxo-1,2-benzisothiazol-2(3H)-yl)phenyl]propanoic acid;

2-[4-(1,3-dioxo-1,3-dihydro-2H-isoindol-2-yl)phenyl]propanoic acid.

[4-(1,3-dioxo-1,3-dihydro-2H-isoindol-2-yl)phenyl]acetic acid;

[4-(1-oxooctahydro-2H-isoindol-2-yl)phenyl]acetic acid;

[4-(2,5-dioxo-2,5-dihydro-1H-pyrrol-1-yl)phenyl]acetic acid;

[4-(1,1-dioxido-3-oxo-1,2-benzisothiazol-2(3H)-yl)phenyl]acetic acid;

[4-(1H-pyrrol-1-yl)phenyl]acetic acid;

[4-(7-hydroxy-3H-[1,2,3]triazolo[4,5-d]pyrimidin-3-yl)phenyl]acetic acid;

[2-chloro-4-(1-oxo-1,3-dihydro-2H-isoindol-2-yl)phenyl]acetic acid.

1) A method for treating cell proliferative disorders associated with an altered cell cycle dependent kinase activity, by administering to a mammal in need thereof an effective amount of a phenylacetamido-pyrazole derivative represented by formula (I): 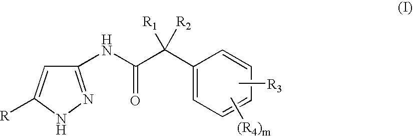
wherein
R is an optionally substituted C₃-C₅ cycloalkyl group;
R₁ and R₂, the same or different, represent hydrogen, halogen, amino, hydroxy or a group selected from straight or branched C₁-C₅ alkyl optionally substituted by amino or hydroxy, straight or branched C₁-C₅ perfluorinated alkyl or straight or branched C₁-C₅ alkoxy or, taken together with the carbon atom to which they are bonded, R₁ and R₂ form an exomethylene (>C═CH₂) group or a C₃-C₄ cycloalkyl group;
R₃, in position 3 or 4 of the phenyl ring, is a group of formula 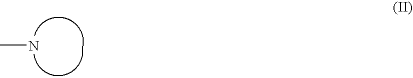
representing a 5 or 6 membered saturated or unsaturated nitrogen containing heterocycle; optionally containing from 1 to 2 additional heteroatoms, the same or different, selected from nitrogen, oxygen or sulfur; optionally substituted in any of the free positions by one or more groups selected from halogen; hydroxy; aminocarbonyl; C₁-C₄ alkylaminocarbonyl; oxo groups (>C═O, >S═O, >SO₂); exomethylene (>C═CH₂); straight or branched C₁-C₄ alkyl, perfluorinated alkyl, hydroxyalkyl or alkoxy groups; C₂-C₄ alkenyl or aryl groups; optionally condensed with carbocyclic or heterocyclic rings, either saturated or unsaturated, either monocyclic or bicyclic, each of which being optionally further substituted as above defined;
m is 0 or an integer from 1 to 4;
if present, each R₄ is, the same or different, halogen, hydroxy or a group selected from straight or branched C₁-C₄ alkyl, perfluorinated alkyl or alkoxy;
or a pharmaceutically acceptable salt thereof;
provided that:
a) when R is cyclopropyl and R₁ and R₂ are both hydrogen atoms, then the nitrogen containing heterocycle of formula (II) is other than 1-pyrrolidinyl, 2-oxo-1-pyrrolidinyl or 1,2,3-triazol-1-yl; and
b) when R is cyclopropyl, one of R₁ and R₂ is a hydrogen atom and the other is methyl, ethyl, n-propyl or n-butyl, then the nitrogen-containing heterocycle of formula (II) is other than 1,3-dihydro-2H-isoindol-2-yl or 1-oxo-1,3-dihydro-2H-isoindol-2-yl.

2) The method according to claim 1 wherein the cell proliferative disorder is selected from the group consisting of cancer, Alzheimer's disease, viral infections, auto-immune diseases and neurodegenerative disorders.

3) The method according to claim 2 wherein the cancer is selected from the group consisting of carcinoma, squamous cell carcinoma, hematopoietic tumors of myeloid or lymphoid lineage, tumors of mesenchymal origin, tumors of the central and peripheral nervous system, melanoma, seminoma, teratocarcinoma, osteosarcoma, xeroderma pigmentosum, keratoxanthoma, thyroid follicular cancer, and Kaposi's sarcoma.

4) The method according to claim 1 wherein the cell proliferative disorder is selected from the group consisting of benign prostate hyperplasia, familial adenomatosis polyposis, neuro-fibromatosis, psoriasis, vascular smooth cell proliferation associated with atherosclerosis, pulmonary fibrosis, arthritis, glomerulonephritis and post-surgical stenosis and restenosis.

5) The method according to claim 1 which provides tumor angiogenesis and metastasis inhibition.

6) The method according to claim 1 which provides treatment or prevention of radiotherapy-induced or chemotherapy-induced alopecia.

7) The method according to claim 1 further comprising subjecting the mammal in need thereof to a radiation therapy or chemotherapy regimen in combination with at least one cytostatic or cytotoxic agent.

8) The method according to claim 1 wherein the mammal in need thereof is a human.

9) The method according to claim 1 which comprises administering to a mammal in need thereof an effective amount of a phenylacetamido-pyrazole derivative represented by formula (I′): 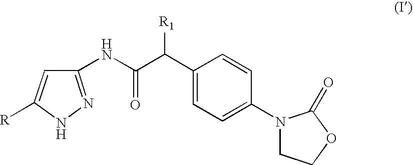
wherein R is a C₃-C₅ cycloalkyl group, R₁ is a hydrogen atom or a methyl group; or a pharmaceutically acceptable salt thereof.

10) The method according to claim 9 wherein the compound of formula (I′) is (2S)-N-(5-cyclopropyl-1H-pyrazol-3-yl)-2-[4-(2-oxo-1,3-oxazolidin-3-yl)phenyl]propanamide.

11) A method for inhibiting cyclin dependent kinase activity which comprises contacting the said kinase with an effective amount of a compound of formula (I) as defined in claim 1.

12) A phenylacetamido-pyrazole derivative represented by formula (I): 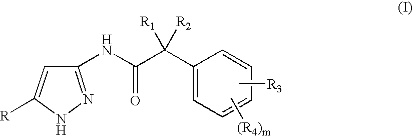
wherein
R is an optionally substituted C₃-C₅ cycloalkyl group;
R₁ and R₂, the same or different, represent hydrogen, halogen, amino, hydroxy or a group selected from straight or branched C₁-C₅ alkyl optionally substituted by amino or hydroxy, straight or branched C₁-C₅ perfluorinated alkyl or straight or branched C₁-C₅ alkoxy or, taken together with the carbon atom to which they are bonded, R₁ and R₂ form an exomethylene (>C═CH₂) group or a C₃-C₄ cycloalkyl group;
R₃, in position 3 or 4 of the phenyl ring, is a group of formula 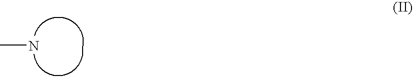
representing a 5 or 6 membered saturated or unsaturated nitrogen containing heterocycle; optionally containing from 1 to 2 additional heteroatoms, the same or different, selected from nitrogen, oxygen or sulfur; optionally substituted in any of the free positions by one or more groups selected from halogen; hydroxy; aminocarbonyl; C₁-C₄ alkylaminocarbonyl; oxo groups (>C═O, >S═O, >SO₂); exomethylene (>C═CH₂); straight or branched C₁-C₄ alkyl, perfluorinated alkyl, hydroxyalkyl or alkoxy groups; C₂-C₄ alkenyl or aryl groups; optionally condensed with carbocyclic or heterocyclic rings, either saturated or unsaturated, either monocyclic or bicyclic, each of which being optionally further substituted as above defined;
m is 0 or an integer from 1 to 4;
if present, each R₄ is, the same or different, halogen, hydroxy or a group selected from straight or branched C₁-C₄ alkyl, perfluorinated alkyl or alkoxy;
or a pharmaceutically acceptable salt thereof;
provided that:
a) when R is cyclopropyl and R₁ and R₂ are both hydrogen atoms, then the nitrogen containing heterocycle of formula (II) is other than 1-pyrrolidinyl, 2-oxo-1-pyrrolidinyl or 1,2,3-triazol-1-yl; and
b) when R is cyclopropyl, one of R₁ and R₂ is a hydrogen atom and the other is methyl, ethyl, n-propyl or n-butyl, then the nitrogen-containing heterocycle of formula (II) is other than 1,3-dihydro-2H-isoindol-2-yl or 1-oxo-1,3-dihydro-2H-isoindol-2-yl.

13) A phenylacetamido-pyrazole derivative of formula (I), according to claim 12, wherein R is a C₃-C₅ cycloalkyl group; one of R₁ and R₂ is hydrogen and the other is a halogen atom or a straight or branched C₁-C₄ alkyl, perfluorinated alkyl or aminoalkyl group; and R₃, R₄ and m have the meanings reported in claim 12.

14) A phenylacetamido-pyrazole derivative of formula (I), according to claim 12, wherein R is a C₃-C₅ cycloalkyl group; R₁ and R₂ are both halogen atoms or taken together with the carbon atom to which they are bonded form an exomethylene group or a C₃-C₄ cycloalkyl group; and R₃, R₄ and m have the meanings reported in claim 12.

15) A phenylacetamido-pyrazole derivative of formula (I), according to claim 12 wherein R₁ and R₂ are both fluorine atoms, and R₃, R₄ and m have the meanings reported in claim 12.

16) A phenylacetamido-pyrazole derivative of formula (I), according to claim 12, wherein R, R₁, R₂, R₄ and m are as defined in claim 12 and R₃, being optionally further substituted and/or condensed, is selected from the group consisting of: 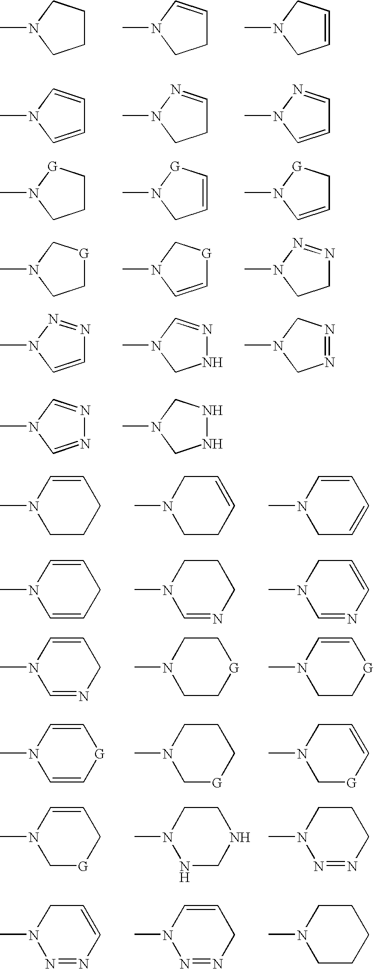
wherein G represents a group —NH—, —O—, —S— or —SO₂—.

17) A phenylacetamido-pyrazole derivative of formula (I), according to claim 16, wherein R₃ is selected from the group consisting of: 1-pyrrolidinyl; 2-oxo-1-pyrrolidinyl; 3-methyl-2-oxo-1-pyrrolidinyl; 2-methyl-5-oxo-1-pyrrolidinyl; 2-ethyl-5oxo-1-pyrrolidinyl; 2-oxo-5-phenyl-1-pyrrolidinyl; 2-oxo-1,3-oxazolidin-3-yl; 2-oxo-3,3a,6,6a-tetrahydrocyclopenta-[b]pyrrol-1(2H)-yl; 2-(hydroxymethyl)-5-oxo-1-pyrrolidinyl; 3-hydroxy-2-oxo-1-pyrrolidinyl; 4-hydroxy-2-oxo-1-pyrrolidinyl; 3-hydroxy-4,4-dimethyl-2-oxo-1-pyrrolidinyl; 2-oxo-3-pyrrolidinecarboxamide; 5-oxo-3-pyrrolidinecarboxamide; 5-oxo-2-pyrrolidinecarboxamide; 1,3-dioxo-1,3-dihydro-2H-isoindol-2-yl; 1-hydroxy-3-oxo-1,3-dihydro-2H-isoindol-2-yl; 1-oxooctahydro-2H-isoindol-2-yl; 2,5-dioxo-2,5-dihydro-1H-pyrrol-1-yl; 2,5-dioxo-1-pyrrolidinyl; 2,5-dioxo-1-pyrrolidinyl; 6-methoxy-1-oxo-1,3-dihydro-2H-isoindol-2-yl; 1,1-dioxido-3-oxo-1,2-benzisothiazol-2(3H)-yl; 1,3-dioxo-1,3-dihydro-2H-benzo[f]isoindol-2-yl; 1-oxo-1H-benzo[de]isoquinolin-2(3H)-yl; 1H-pyrrol-1-yl; 7-hydroxy-3H-[1,2,3]triazolo[4,5-d]pyrimidin-3-yl; 3H-[1,2,3]triazolo[4,5-d]pyrimidin-3-yl; 1-oxo-1,3-dihydro-2H-isoindol-2-yl; 2,4-dioxotetrahydro-1(2H)-pyrimidinyl; 3,5-dioxo-1,2,4-triazolidin-4-yl; 2,5-dioxo-1-imidazolidinyl; 2,4-dioxo-1-imidazolidinyl; 2-oxo-1-imidazolidinyl; 2-oxo-2,3-dihydro-1H-imidazol-1-yl; 2-oxo-2,3-dihydro-1H-imidazol-1-yl; 5-oxo-1,5-dihydro-4H-1,2,4-triazol-4-yl; 5-oxo-1,5-dihydro-4H-1,2,4-triazol-4-yl; 5-hydroxy-1H-pyrazole-3-carboxamide; 3-oxo-1-pyrazolidinyl; 3,5-dioxo-1-pyrazolidinyl; 2,4-dioxo-3,4-dihydro-1(2H)-pyrimidinyl; 3,5-dioxo-4-morpholinyl; 2-oxo-1-piperidinyl; 1-piperazinyl; 4-morpholinyl and 1-piperidinyl.

18) A phenylacetamido-pyrazole derivative of formula (I), according to claim 12, which is represented by formula (I′) 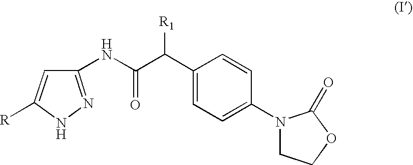
wherein
R is a C₃-C₅ cycloalkyl group;
R₁ is a hydrogen atom or a methyl group;
and the pharmaceutically acceptable salts thereof.

19) A phenylacetamido-pyrazole derivative of formula (I′), according to claim 18, wherein R is cyclopropyl.

20) A phenylacetamido-pyrazole derivative of formula (I′), according to claim 18, wherein R₁ is methyl.

21) A phenylacetamido-pyrazole derivative of formula (I′), according to claim 18, wherein R is cyclopropyl and R₁ is methyl.

22) The phenylacetamido-pyrazole derivative of formula (I′), according to claim 21, which is (2S)-N-(5-cyclopropyl-1H-pyrazol-3-yl)-2-[4-(2-oxo-1,3-oxazolidin-3-yl)phenyl]propanamide.

23) A phenylacetamido-pyrazole derivative of formula (I), according to claim 12, optionally in the form of a pharmaceutically acceptable salt, selected from the group consisting of:
1. N-(5-cyclopropyl-1H-pyrazol-3-yl)-2-[4-(2-oxo-1,3-oxazolidin-3-yl)phenyl]propanamide;
2. (2R)-N-(5-cyclopropyl-1H-pyrazol-3-yl)-2-[4-(2-oxo-1,3-oxazolidin-3-yl)phenyl]propanamide;
3. (2S)-N-(5-cyclopropyl-1H-pyrazol-3-yl)-2-[4-(2-oxo-1,3-oxazolidin-3-yl)henyl]propanamide;
4. N-(5-cyclopropyl-1H-pyrazol-3-yl)-2-[4-(2-oxo-1,3-oxazolidin-3-yl)phenyl]acetamide;
5. N-(5-cyclobutyl-1H-pyrazol-3-yl)-2-[4-(2-oxo-1,3-oxazolidin-3-yl)phenyl]propanamide;
6. (2R)-N-(5-cyclobutyl-1H-pyrazol-3-yl)-2-[4-(2-oxo-1,3-oxazolidin-3-yl)phenyl]propanamide;
7. (2S)-N-(5-cyclobutyl-1H-pyrazol-3-yl)-2-[4-(2-oxo-1,3-oxazolidin-3-yl)phenyl]propanamide;
8. N-(5-cyclobutyl-1H-pyrazol-3-yl)-2-[4-(2-oxo-1,3-oxazolidin-3-yl)phenyl]acetamide;
9. N-(5-cyclopentyl-1H-pyrazol-3-yl)-2-[4-(2-oxo-1,3-oxazolidin-3-yl)phenyl]propanamide;
10. (2R)-N-(5-cyclopentyl-1H-pyrazol-3-yl)-2-[4-(2-oxo-1,3-oxazolidin-3-yl)phenyl]propanamide;
11. (2S)-N-(5-cyclopenyl-1H-pyrazol-3-yl)-2-[4-(2-oxo-1,3-oxazolidin-3-yl)phenyl]propanamide;
12. N-(5-cyclopentyl-1H-pyrazol-3-yl)-2-[4-(2-oxo-1,3-oxazolidin-3-yl)phenyl]acetamide;
13. N-(5-cyclopropyl-1H-pyrazol-3-yl)-2-[4-(2-oxo-1-pyrrolidinyl)phenyl]propanamide;
14. (2S)-N-(5-cyclopropyl-1H-pyrazol-3-yl)-2-[4-(2-oxo-1-pyrrolidinyl)phenyl]propanamide;
15. (2R)-N-(5-cyclopropyl-1H-pyrazol-3-yl)-2-[4-(2-oxo-1-pyrrolidinyl)phenyl]propanamide;
16. N-(5-cyclobutyl-1H-pyrazol-3-yl)-2-[4-(2-oxo-1-pyrrolidinyl)phenyl]propanamide;
17. (2S)-N-(5-cyclobutyl-1H-pyrazol-3-yl)-2-[4-(2-oxo-1-pyrrolidinyl)phenyl]propanamide;
18. (2R)-N-(5-cyclobutyl-1H-pyrazol-3-yl)-2-[4-(2-oxo-1-pyrrolidinyl)phenyl]propanamide;
19. N-(5-cyclopropyl-1H-pyrazol-3-yl)-2-fluoro-2-[4-(2-oxo-1-pyrrolidinyl)phenyl]acetamide;
20. 2-chloro-N-(5-cyclopropyl-1H-pyrazol-3-yl)-2-[4-(2-oxo-1-pyrrolidinyl)phenyl]acetamide;
21. N-(5-cyclopropyl-1H-pyrazol-3-yl)-2,2-difluoro-2-[4-(2-oxo-1-pyrrolidinyl)phenyl]acetamide;
22. N-(5-cyclopropyl-1H-pyrazol-3-yl)-3,3,3-trifluoro-2-[4-(2-oxo-1-pyrrolidinyl)phenyl]propanamide;
23. 3-amino-N-(5-cyclopropyl-1H-pyrazol-3-yl)-2-[4-(2-oxo-1-pyrrolidinyl)phenyl]propanamide;
24. N-(5-cyclopropyl-1H-pyrazol-3-yl)-2-[4-(3-methyl-2-oxo-1-pyrrolidinyl)phenyl]acetamide;
25. N-(5-cyclopropyl-1H-pyrazol-3-yl)-2-[4-(3-methyl-2-oxo-1-pyrrolidinyl)phenyl]propanamide;
26. N-(5-cyclopropyl-1H-pyrazol-3-yl)-2-[4-(2-methyl-5-oxo-1-pyrrolidinyl)phenyl]acetamide;
27. N-(5-cyclopropyl-1H-pyrazol-3-yl)-2-[4-(2-methyl-5-oxo-1-pyrrolidinyl)phenyl]propanamide;
28. N-(5-cyclopropyl-1H-pyrazol-3-yl)-2-[4-(2-ethyl-5-oxo-1-pyrrolidinyl)phenyl]acetamide;
29. N-(5-cyclopropyl-1H-pyrazol-3-yl)-2-[4-(2-ethyl-5-oxo-1-pyrrolidinyl)phenyl]propanamide;
30. N-(5-cyclopropyl-1H-pyrazol-3-yl)-2-[4-(2-oxo-5-phenyl-1-pyrrolidinyl)phenyl]acetamide;
31. N-(5-cyclopropyl-1H-pyrazol-3-yl)-2-[4-(2-oxo-5-phenyl-1-pyrrolidinyl)phenyl]propanamide;
32. N-(5-cyclopropyl-1H-pyrazol-3-yl)-2-[4-(2-oxo-3,3a,6,6a-tetrahydrocyclopenta[b]pyrrol-1(2H)-yl)phenyl]acetamide;
33. N-(5-cyclopropyl-1H-pyrazol-3-yl)-2-[4-(2-oxo-3,3a,6,6a-tetrahydrocyclopenta[b]pyrrol-1(2H)-yl)phenyl]propanamide;
34. N-(5-cyclopropyl-1H-pyrazol-3-yl)-2-{4-[2-(hydroxymethyl)-5-oxo-1-pyrrolidinyl]phenyl}acetamide;
35. N-(5-cyclopropyl-1H-pyrazol-3-yl)-2-{4-[2-(hydroxymethyl)-5-oxo-1-pyrrolidinyl]phenyl}propanamide;
36. N-(5-cyclopropyl-1H-pyrazol-3-yl)-2-[4-(3-hydroxy-2-oxo-1-pyrrolidinyl)phenyl]acetamide;
37. N-(5-cyclopropyl-1H-pyrazol-3-yl)-2-[4-(3-hydroxy-2-oxo-1-pyrrolidinyl)phenyl]propanamide;
38. N-(5-cyclopropyl-1H-pyrazol-3-yl)-2-[4-(4-hydroxy-2-oxo-1-pyrrolidinyl)phenyl]acetamide;
39. N-(5-cyclopropyl-1H-pyrazol-3-yl)-2-[4-(4-hydroxy-2-oxo-1-pyrrolidinyl)phenyl]propanamide;
40. N-(5-cyclopropyl-1H-pyrazol-3-yl)-2-[4-(3-hydroxy-4,4-dimethyl-2-oxo-1-pyrrolidinyl)phenyl]acetamide;
41. N-(5-cyclopropyl-1H-pyrazol-3-yl)-2-[4-(3-hydroxy-4,4-dimethyl-2-oxo-1-pyrrolidinyl)phenyl]propanamide;
42. 1-(4-{2-[(5-cyclopropyl-1H-pyrazol-3-yl)amino]-2-oxoethyl}phenyl)-2-oxo-3-pyrrolidinecarboxamide;
43. 1-(4-{2-[(5-cyclopropyl-1H-pyrazol-3-yl)amino]-1-methyl-2-oxoethyl}phenyl)-2-oxo-3-pyrrolidinecarboxamide;
44. 1-(4-{2-[(5-cyclopropyl-1H-pyrazol-3-yl)amino]-2-oxoethyl}phenyl)-5-oxo-3-pyrrolidinecarboxamide;
45. 1-(4-{2-[(5-cyclopropyl-1H-pyrazol-3-yl)amino]-1-methyl-2-oxoethyl}phenyl)-5-oxo-3-pyrrolidinecarboxamide;
46. 1-(4-{2-[(5-cyclopropyl-1H-pyrazol-3-yl)amino]-2-oxoethyl}phenyl)-5-oxo-2-pyrrolidinecarboxamide;
47. 1-(4-{2-[(5-cyclopropyl-1H-pyrazol-3-yl)amino]-1-methyl-2-oxoethyl}phenyl)-5-oxo-2-pyroolidinecarboxamide;
48. N-(5-cyclopropyl-1H-pyrazol-3-yl)-2-[4-(1,3-dioxo-1,3-dihydro-2H-isoindol-2-yl)phenyl]acetamide;
49. N-(5-cyclopropyl-1H-pyrazol-3-yl)-2-[4-(1,3-dioxo-1,3-dihydro-2H-isoindol-2-yl)phenyl]propanamide;
50. N-(5-cyclopropyl-1H-pyrazol-3-yl)-2-[4-(1-hydroxy-3-oxo-1,3-dihydro-2H-isoindol-2-yl)phenyl]propanamide;
51. N-(5-cyclopropyl-1H-pyrazol-3-yl)-2-[4-(1-oxooctahydro-2H-isoindol-2-yl)phenyl]acetamide;
52. N-(5-cyclopropyl-1H-pyrazol-3-yl)-2-[4-(1-oxooctahydro-2H-isoindol-2-yl)phenyl]propanamide;
53. N-(5-cyclopropyl-1H-pyrazol-3-yl)-2-[4-(2,5-dioxo-2,5-dihydro-1H-pyrrol-1-yl)phenyl]acetamide;
54. N-(5-cyclopropyl-1H-pyrazol-3-yl)-2-[4-(2,5-dioxo-2,5-dihydro-1H-pyrrol-1-yl)phenyl]propanamide;
55. N-(5-cyclopropyl-1H-pyrazol-3-yl)-2-[4-(2,5-dioxo-1-pyrrolidinyl)phenyl]acetamide;
56. N-(5-cyclopropyl-1H-pyrazol-3-yl)-2-[4-(2,5-dioxo-1-pyrrolidinyl)phenyl]propanamide;
57. N-(5-cyclopropyl-1H-pyrazol-3-yl)-2-[4-(6-methoxy-1-oxo-1,3-dihydro-2H-isoindol-2-yl)phenyl]propanamide;
58. N-(5-cyclopropyl-1H-pyrazol-3-yl)-2-[4-(1,1-dioxido-3-oxo-1,2-benzisothiazol-2(3H)-yl)phenyl]acetamide;
59. N-(5-cyclopropyl-1H-pyrazol-3-yl)-2-[4-(1,1-dioxido-3-oxo-1,2-benzisothiazol-2(3H)-yl)phenyl]propanamide;
60. N-(5-cyclopropyl-1H-pyrazol-3-yl)-2-[4-(1,3-dioxo-1,3-dihydro-2H-benzo[f]isoindol-2-yl)phenyl]propanamide;
61. N-(5-cyclopropyl-1H-pyrazol-3-yl)-2-[4-(1-oxo-1H-benzo[de]isoquinolin-2(3H)-yl)phernyl]propanamide;
62. N-(5-cyclopropyl-1H-pyrazol-3-yl)-2-[4-(1H-pyrrol-1-yl)phenyl]acetamide;
63. N-(5-cyclopropyl-1H-pyrazol-3-yl)-2-[4-(1H-pyrrol-1-yl)phenyl]propanamide;
64. N-(5-cyclopropyl-1H-pyrazol-3-yl)-2-[4-(7-hydroxy-3H-[ 1,2,3]triazolo[4,5d]pyrimidin-3-yl)phenyl]acetamide;
65. N-(5-cyclopropyl-1H-pyrazol-3-yl)-2-[4-(3H-[1,2,3]triazolo[4,5-d]pyrimidin-3-yl)phenyl]propanamide;
66. 2-[2-chloro-4-(1-oxo-1,3-dihydro-2H-isoindol-2-yl)phenyl]-N-(5-cyclopropyl-1H-pyrazol-3-yl)aetamide;
67. 2-[2-chloro-4-(1-oxo-1,3-dihydro-2H-isoindol-2-yl)phenyl]-N-(5-cyclopropyl-1H-pyrazol-3-yl)propanamide;
68. N-(5-cyclopropyl-1H-pyrazol-3-yl)-2-[4-(2,4-dioxotetrahydro-1(2H)-pyrimidinyl)phenyl]acetamide;
69. N-(5-cyclopropyl-1H-pyrazol-3-yl)-2-[4-(2,4-dioxotetrahydro-1(2H)-pyrimidinyl)phenyl]acetamide;
70. N-(5-cyclopropyl-1H-pyrazol-3-yl)-2-[4-(3,5-dioxo-1,2,4-triazolidin-4-yl)phenyl]acetamide;
71. N-(5-cyclopropyl-1H-pyrazol-3-yl)-2-[4-(3,5-dioxo-1,2,4-triazolidin-4-yl)phenyl]propanamide;
72. N-(5-cyclopropyl-1H-pyrazol-3-yl)-2-[4-(2,5-dioxo 1-imidazolidinyl)phenyl]acetamide;
73. N-(5-cyclopropyl-1H-pyrazol-3-yl)-2-[4-(2,5-dioxo 1-imidazolidinyl)phenyl]propanamide;
74. N-(5-cyclopropyl-1H-pyrazol-3-yl)-2-[4-(2,4-dioxo 1-imidazolidinyl)phenyl]acetamide;
75. N-(5-cyclopropyl-1H-pyrazol-3-yl)-2-[4-(2,4-dioxo 1-imidazolidinyl)phenyl]propanamide;
76. N-(5-cyclopropyl-1H-pyrazol-3-yl)-2-[4-(2-oxo 1-imidazolidinyl)phenyl]acetamide;
77. N-(5-cyclopropyl-1H-pyrazol-3-yl)-2-[4-(2-oxo 1-imidazolidinyl)phenyl]propanamide;
78. N-(5-cyclopropyl-1H-pyrazol-3-yl)-2-[4-(2-oxo-2,3-dihydro-1H-imidazol-1-yl)phenyl]acetamide;
79. N-(5-cyclopropyl-1H-pyrazol-3-yl)-2-[4-(2-oxo-2,3-dihydro-1H-imidazol-1-yl)phenyl]propanamide;
80. N-(5-cyclopropyl-1H-pyrazol-3-yl)-2-[4-(5-oxo-1,5-dihydro-4H-1,2,4-triazol-4-yl)phenyl]acetamide;
81. N-(5-cyclopropyl-1H-pyrazol-3-yl)-2-[4-(5-oxo-1,5-dihydro-4H-1,2,4-triazol-4-yl)phenyl]propanamide;
82. 1-(4-{2-[(5-cyclopropyl-1H-pyrazol-3-yl)amino]-2-oxoethyl}phenyl)-5-hydroxy-1H-pyroazole-3-carboxamide;
83. 1-(4-{2-[(5-cyclopropyl-1H-pyrazol-3-yl)amino]-1-methyl-2-oxoethyl}phenyl)-5-hydroxy-1H-pyrazole-3-carboxamide;
84. N-(5-cyclopropyl-1H-pyrazol-3-yl)-2-[4-(3-oxo-1-pyrazolidinyl)phenyl]acetamide;
85. N-(5-cyclopropyl-1H-pyrazol-3-yl)-2-[4-(3-oxo-1-pyrazolidinyl)phenyl]propanamide;
86. N-(5-cyclopropyl-1H-pyrazol-3-yl)-2-[4-(3,5-dioxo-1-pyrazolidinyl)phenyl]acetamide;
87. N-(5-cyclopropyl-1H-pyrazol-3-yl)-2-[4-(3,5-dioxo-1-pyrazolidinyl)phenyl]propanamide;
88. N-(5-cyclopropyl-1H-pyrazol-3-yl)-2-[4-(2,4-dioxo-3,4-dihydro-1(2H)-pyrimidinyl)phenyl]acetamide;
89. N-(5-cyclopropyl-1H-pyrazol-3-yl)-2-[4-(2,4-dioxo-3,4-dihydro-1(2H)-pyrimidinyl)phenyl]propanamide;
90. N-(5-cyclopropyl-1H-pyrazol-3-yl)-2-[4-(3,5-dioxo-4-morpholinyl)phenyl]acetamide;
91. N-(5-cyclopropyl-1H-pyrazol-3-yl)-2-[4-(3,5-dioxo-4-morpholinyl)phenyl]propanamide;
92. N-(5-cyclopropyl-1H-pyrazol-3-yl)-2-[4-(2-oxo-1-piperidinyl)phenyl]acetamide;
93. N-(5-cyclopropyl-1H-pyrazol-3-yl)-2-[4-(2-oxo-1-piperidinyl)phenyl]propanamide;
94. N-(5-cyclopropyl-1H-pyrazol-3-yl)-2-[4-(1-piperazinyl)phenyl]acetamide;
95. N-(5-cyclopropyl-1H-pyrazol-3-yl)-2-[4-(1-piperazinyl)phenyl]propanamide;
96. N-(5-cyclopropyl-1H-pyrazol-3-yl)-2-[4-(4-morpholinyl)phenyl]acetamide;
97. N-(5-cyclopropyl-1H-pyrazol-3-yl)-2-[4-(4-morpholinyl)phenyl]propanamide;
98. N-(5-cyclopropyl-1H-pyrazol-3-yl)-2-[4-(1-piperidinyl)phenyl]acetamide;
99. N-(5-cyclopropyl-1H-pyrazol-3-yl)-2-[4-(1-pyrrolidinyl)phenyl]propanamide;
100. N-(5-cyclopropyl-1H-pyrazol-3-yl)-2-[4-(1-piperidinyl)phenyl]propanamide.

24) A process for preparing the compounds of formula (I) or the pharmaceutically acceptable salts thereof, as defined in claim 12, which process comprises:
a) reacting the compounds of formula (III) or the regioisomers of formula (IIIa) 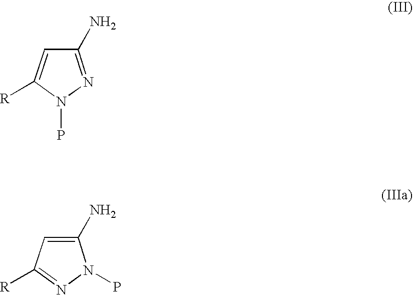
wherein R is as defined in claim 12 and P represents a nitrogen-pyrazole protecting group, with the compounds of formula (IV) 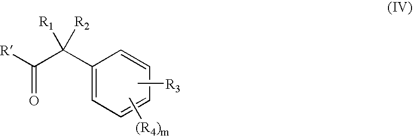
wherein R₁, R₂, R₃, R₄ and m are as defined in claim 12 and R′ represents hydroxy or a halogen atom, thus obtaining the compounds of formula (V) or (Va) 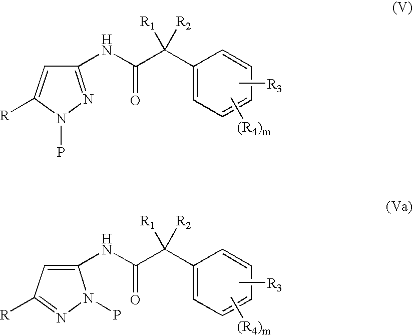
b) and deprotecting the compounds of formula (V) or (Va) so as to obtain the derivatives of formula (I) and, if desired, converting them into pharmaceutically acceptable salts thereof.

25) The process of claim 24 wherein, within the compounds of formula (III) or (IIIa), P represents the group tert-butoxycarbonyl (boc).

26) The process of claim 24 wherein, within the compounds of formula (IV), R′ is hydroxy or a chlorine atom.

27) A process for preparing the compounds of formula (I′) and the pharmaceutically acceptable salts, as defined in claim 18, which process comprises:
a) reacting the compounds of formula (XXXV) 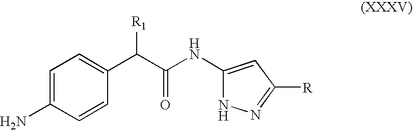
wherein R and R₁ are as defined in claim 18, with chloroethylchloroformate in the presence of a base, thus obtaining the compounds of formula (XXXVI) 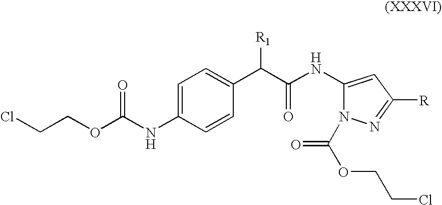
b) reacting the compounds of formula (XXXVI) with a suitable base, thus obtaining the compounds of formula (XXXVII) 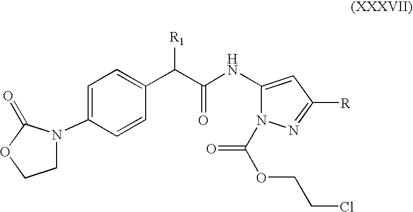
c) and hydrolyzing under basic conditions the compounds of formula (XXXVII) so as to obtain the desired compounds of formula (I′) and, if desired, converting them into pharmaceutically acceptable salts thereof.

28) The process of claim 27 wherein step a) is carried out in the presence of a base selected from the group consisting of pyridine, triethylamine or N,N-diisopropylethylamine.

29) The process of claim 27 wherein step b) is carried out in the presence of a base selected from potassium carbonate or 1,8-diazabicyclo[5.4.0]undec-7-ene.

30) A pharmaceutical composition comprising a theraputically effective amount of a phenylacetamido-pyrazole derivative of formula (I), as defined in claim 12, and at least one pharmaceutically acceptable excipient, carrier and/or diluent.

31) A pharmaceutical composition according to claim 30 further comprising one or more chemotherapeutic agents, as a combined preparation for simultaneous, separate or sequential use in anticancer therapy.

32) A product or kit comprising a compound of formula (I) or a pharmaceutically acceptable salt as defined in claim 12 or a pharmaceutical composition thereof, as defined in claim 30, and one or more chemotherapeutic agents, as a combined preparation for simultaneous, separate or sequential use in anticancer therapy.

33) A compound of formula (I) or a pharmaceutically acceptable salt thereof, as defined in claim 12, for us as a medicament.

34) Use of a compound of formula (I) or a pharmaceutically acceptable salt thereof, as defined in claim 12, in the manufacture of a medicament with cell cycle dependent kinase inhibitory activity.

35) Use of a compound of formula (I) or a pharmaceutically acceptable salt thereof, as defined in claim 12, in the manufacture of a medicament with antitumor activity.
